(19) United States
(12) Patent Application Publication
(10) Pub. No.: US 2020/0390360 A1
(43) Pub. Date: Dec. 17, 2020

(54) Automatic Machine for Identifying Victim of Abuse Voice to Skull and Remote Neural Monitoring Technology and for Identifying Remote Attacker or Operator Using Device of Voice to Skull and Remote Neural Monitoring

(71) Applicant: Da LI, West Covina, CA (US)

(72) Inventor: Da LI, West Covina, CA (US)

(21) Appl. No.: 16/997,656

(22) Filed: Aug. 19, 2020

Related U.S. Application Data
(63) Continuation-in-part of application No. 16/558,040, filed on Aug. 31, 2019.
(60) Provisional application No. 62/812,915, filed on Mar. 1, 2019.

(51) Int. Cl. A61B 5/0484; G06F 3/0488; G06F 3/14; A61B 5/00; G16H 40/67; G06F 16/44; G16H 20/70

(52) U.S. Cl. CPC A61B 5/04842; G06F 3/0488; G06F 3/14; G16H 20/70; G16H 40/67; G06F 16/44; A61B 5/742

(57) ABSTRACT

Automatic machine can both identify victim and remote operator using device of V2K and Remote Neural Monitoring. The automatic machine using databases consisting seven catalogs contents. To launching a retinal image signal attacking remote operator, ask a tested person select and watch one image or video with some words attacking remote operator only causing remote operator's psychological response, but doesn't cause tested person's psychological response, then answers question on a touchscreen according V2K feedback signals by pressing buttons YES or NO, the automatic identifying system will show “CORRECT” or “WRONG” with every pressing. This function can guide the tested person to get final accurate identification quickly. Meanwhile printing and storing system can print certificate to victim and documents of all kinds status of remote operator, store them in an information database in computer; provide an access to users in network for updating information.

### CROSS REFERENCE OF RELATED APPLICATION

This is a Continuation-In-Part application of a non-provisional application Ser. No. 16/558,040, filing date Aug. 31, 2019, which claims the benefit of U.S. provisional patent application No. 62/812,915, filing date Mar. 1, 2019. The content of these specifications, including any intervening amendments thereto, are incorporated by reference herein in their entirety.

### BACKGROUND OF THE PRESENT INVENTION

### Field of Invention

The present invention relates to a kind of automatic machine can both identify who is victim of abuse voice to skull and remote neural monitoring technology and identify who is remote operator using device of voice to skull and remote neural monitoring. Then produce a certificate to a identified victim for medical department using, meanwhile produce some documents about remote attacker or operator's status submitting to department of justice for reference.

### Description of Related Arts

Origin of the problem: from 2008 to 2018, there were large number of victims in 22 provinces in China, Shanghai and Beijing municipalities have large number of collective protests against RNM and V2K technology abuse. This is also a part of operational level of war or called unrestricted war which China has somehow partly launched to attack US and other countries now or in no long future.

From psychiatric taxonomy analysis, only rare case of hysteria likely to occur in group under special situation. These same groups of large-scale protester's peaceful and rational activities year and year in China are not hysteria patients obviously.

In addition, on Oct. 1, 2015, representatives from 17 countries in Berlin, Germany, hold a “Covered Harassment Conference”. The covered harassment is same thing with voice to skull & remote neural monitoring technology.

After a lot of times contacting with these victims in China mainland WeChat, emails and telephones, these victims' thoughts were read and harassed at same time by some unknown Chinese institutions abusing these technologies. Meanwhile all victims know their optic neural image signals, or call retinal cortex image signals and auditory neural signals in their cerebral cortex were monitored remotely.

According to Zhong Zhiyong, the organizer and leader of anti-V2k &RNM torture in 22 provinces and cities in mainland China (Zhong Zhiyong's affairs were also reported by the media “The Epoch Times” and he and other victims representatives also received interview with “New Tang Dynasty TV”). He told me directly by phone and WeChart almost all victims have the same experience: the Remote Neural Monitoring and V2K technology can read victim's auditory signals on cortex and victim's retinal cortex image signals, harass all victims with V2K signals. Including two Chinese victim has been a lawful permanent resident in United States have same experience: their retinal cortex image signals and auditory neural signals in their cerebral cortex were monitored remotely. They are all suffering non-stop V2K harassment.

Another Chinese leader anti-abuse technology of V2K & RNM, Mr. Yao Dou-jie live in Shenzhen China who collect large information also confirmed same cases above.

Jun. 21, 2020, United Nations Human Rights Office of the High Commissioner call for input to a report: Psychosocial dynamics conducive to torture and ill-treatment, decide to investigate and ban electromagnetic Torture, Harrassment techniques torture to individuals. This is first time UN official recognition the existence the abuse V2K and Remote Neural Monitoring Technology.

What is Remote Neural Monitoring Technology? the following US patents describe them clearly: These technology can read human neural signals like human optic neural signals, or call retinal cortex image signals and auditory neural signals in their cerebral cortex to produce harassment to a victim. Because these US patents have external link to European patent database, so all US and European engineers and technical personnel acknowledge the existence of these technologies.

Generally three to five operators (ROD) of V2k & RNM devices in group in turn concentrate one victim via satellite positioning technology to realize remote harassment, the technology is mainly based on these US technological patents and China patents which describe the technology of V2K & RNM are extracting the brain's characteristic wave frequency and their optic neural signals, or call retinal cortex image signals and auditory neural signals in their cerebral cortex first (like fingerprints, each person's brain characteristic wave frequency is different), then codes the brain characteristic wave frequency and these neural signals by computer software technology and remotely locks the frequency and monitors the victim's brain wave frequency and these neural signals using computer software technology and satellite positioning technology.

The bases of these V2K technology are microwave auditory hearing effect or call Frey effect and neural coding technology. The microwave auditory hearing effect is a well-known physical phenomenon and it does not a sound wave shaking eardrum in ear, but directly effects on human auditory cortex with like KAKAKA sound. V2K technology is a kind of improving microwave auditory hearing effect with human language which only a victim can feel, the persons around the victim never hear or feel.

In such way, three to five operators (ROD) using devices of V2K & RNM in groups perform active psychological attacks on victim's brain with some negative emotions according these victim's auditory signal and, retinal cortex image signals shown on the their RNM device. But the effect of this kind of psychological reactions or responses can be two-ways or two directions between victim and operators (RODs) using devices of V2K & RNM.

In past of years, FBI's investigation on some cases related similar technology attacking.

This is not a scientific fiction, because the following US patents and China patents tell us it is true and existing technologies: US patents: All following US patents describe technologies can monitor auditory signals and retinal signals in cerebral cortex remotely.

1.1. U.S. Pat. No. 3,951,134A: Apparatus and method for remotely monitoring and altering brain waves.

Inventor: Robert G. Malech, Date of publication: 1976-04-20 publication

The technology describes such technology can monitor human brain wave and all Kinds neutral signals like auditory signal and retinal signal remotely. Meanwhile cause V2K signal feedback like microwave hearing effect.

Apparatus for and method of sensing brain waves at a position remote from a . . . . The demodulated waveform also can be used to produce a compensating signal . . . . In addition to passively monitoring his brain waves, the subject's neurological signals.

1.2. U.S. Pat. No. 6,470,214B1 Method and device for implementing the radio frequency hearing effect, US Air Force.

1.3. U.S. Pat. No. 7,222,961B2 Method for detecting a functional signal in retinal images.

1.4. U.S. Pat. No. 6,011,991A: Communication system and method including brain wave analysis and/or use of brain activity.

Inventor: Aris Mardirossian; Date of Publication: 2000-01-04

The technology describes how communicate human brain thoughts and cause language like effect remotely via satellite.

A system and method for enabling human beings to communicate by way of their monitored brain activity. The brain activity of an individual is monitored and transmitted to a remote location (e.g. by satellite). At the remote location, the monitored brain activity is compared with pre-recorded normalized brain activity curves, wave forms, or patterns to determine if a match or substantial match is found. If such a match is found, then the computer at the remote location determines that the individual was attempting to communicate the word, phrase, or thought corresponding to the matched stored normalized signal.

1.5. WO2014066598A1, WIPO (PCT): Image retinal intrinsic optical signals Remote neural modulation brain stimulation and feedback control. Inventor: Laura Tyler PERRYMAN, Chad ANDRESEN, Patrick LARSON, Graham GREENE. Date of publication: 2014-09-18.

The method, system and apparatus is presented for a wireless neural modulation feedback control system as it relates to an implantable medical device comprised of a radio frequency (RF) receiver circuit, one or more dipole or patch antenna(s), one or more electrode leads connected to at least one dipole or patch antenna(s), and at least one microelectronic neural modulation circuit, and an external or internally implanted RF device to neurally modulate brain tissue in order to treat medical conditions that can be mediated by neuronal activation or inhibition, such as Parkinson's, Alzheimer's, epilepsy, other motor or mood based disorders, and/or pain. The implantable receiver captures energy radiated by the RF transmitter unit and converts this energy to an electrical waveform by the implanted neural modulation circuit to deliver energy that can be utilized by the attached electrode pads in order to activate targeted neurons in the brain.

1.6. U.S. Pat. No. 8,738,162: Clustering of recorded patient neurological activity to determine length of a neurological event, Inventor: Mark G. Frei, Ivan Osorio, Nina M. Graves, Scott F. Schaffner, Mark T. Rise, Jonathon E. Giftakis, David L. Carlson. Date of Publication: 2014-05-27.

Apparatus and method detect a detection cluster that is associated with a neurological event, such as a seizure, of a nervous system disorder and update therapy parameters that are associated with a treatment therapy. The occurrence of the detection cluster is detected when the maximal ratio exceeds an intensity threshold. If the maximal ratio drops below the intensity threshold for a time interval that is less than a time threshold and subsequently rises above the intensity threshold, the subsequent time duration is considered as being associated with the detection cluster rather than being associated with a different detection cluster. Consequently, treatment of the nervous system disorder during the corresponding time period is in accordance with one detection cluster. Treatment therapy may be provided by providing electrical stimulation, drug infusion or a combination. Therapy parameters may be updated for each mth successive group of applications of the treatment therapy or for each nth detection cluster.

1.7. US20080146960A1, Headset For A Wireless Neural Data Acquisition System, Inventor: Pedro Irazoqui-Pastor, James C. Morizio, Vinson L. Go, Jack D. ParmentireDate of Publication: 2009-12-29.

A headstage for a Neural Data Acquisition System is shown and described. In one embodiment, the headstage includes at least one Input Pre-amplifier, and a multiplexer (MUX) for multiplexing at least one channel. In one embodiment, the input filter of the Input Pre-amplifier is tuned by adjusting the gate voltage of a transistor operating in sub-threshold mode.

1.8. U.S. Pat. No. 4,858,612A Hearing Device, Inventor: Philip L. Stocklin; Date of Publication: 1989-08-22, A method and apparatus for simulation of hearing in mammals by introduction of a plurality of microwaves into the region of the auditory cortex is shown and described. A microphone is used to transform sound signals into electrical signals which are in turn analyzed and processed to provide controls for generating a plurality of microwave signals at different frequencies. The multifrequency microwaves are then applied to the brain in the region of the auditory cortex. By this method sounds are perceived by the ma

1.9. CN 2008202247769.1 China use the tech both in peaceful time and war time.

Publicly published books: All these public books describe some technologies can monitor neural signals like auditory signals and retinal signals in cerebral cortex remotely.

Chinese military research book and public issued books:

2.0. <Control Brain Technology Development and Military Application Prediction and Research> Date of publication November, 2016, Total pages 166—Doctoral Paper of the Third Military University of China Author: Luo Xue, Principle Professors: Gou Jiwei and Liu Cangli.

The doctoral paper shows Chinese military and government institute develop this technology vigorously which is used in Operational Level War to attack our USA and other countries.

2.1. <Neural Monitoring, The Prevention of Intraoperative Injury>, ISBN 978-1-4612-0491-6 Authors: Salzman, Steven K., eBook $219.00

2.2. <Remote Brain Targeting: A Compilation of Historical Data and Information from Various Sources>, eBay:$15.89, Sold by greatbookpricesl (115117) 98.9% Positive feedback

2.3. <Surviving and Thriving as a Targeted Individual: How to Beat Covert Surveillance, Gang Stalking, and Harassment>, eBay: US$19.27 ISBN 10: 1549542931/ISBN 13: 9781549542930

Now we have confirmed these technologies existing with US patents and China patents, as well as publicly issued books.

Till now, there is not any method to identify both a victims of technology abusing and those remote operators (RODs) using devices of V2K and RNM around world. These abuse has been developed global and transnational.

### SUMMARY OF THE PRESENT INVENTION

This invention uses programmable logical controller (PLC) as main comportment, because PLC is widely used in automatic control areas which input all kind signals to be processed by a special CPU inside of PLC. The invention also input all kinds of electronic signals from V2K feedback signals and retinal image signals transformation to achieve identifying goals.

First the invention is used to identify a victim of abuse V2K and RNM technology, the victim receives coded physical electromagnetic and microwave wave radio frequency harassment called V2K harassment feedback signal, V2K's physical base is microwave hearing effect, or called Frey effect, it is an ordinary physical phenomena, Here it needs to be emphasized the V2K signal is generated by voice to skull (V2K) technological device which is described by above US patents, there is not human factor.

Second why the reason the remote operator can harass the victim is the victim's brain wave frequency was taken in very near distance first by someone and then the brain wave frequency was coded and decoded into some software then using satellite communication technology to harass victim remotely and take victim's neural signal such as retinal image signal and auditory cortex signal, like a radio station receive and send shortwave information with fixed frequency. So the invention uses of the remote device taking and showing victim's retinal image signal, with pre-designed images and videos with some text words to display on the remote device of V2K and RNM, deliberately causing the remote operator feedback signals of V2K and RNM V2K. Here also needs to be emphasized the retinal image signal is collected by remote neural monitoring (RNM) technological device which is described by above US patents, there is not human factor.

Because the ROD wants to harass a victim, the ROD must use of his or her negative words with V2K technology based microwave hearing effect to harass the victim via device of V2K and RNM and via satellite communication and obtain victim's emotion reaction from these device, so in this case has a bidirectional and interactive process. The invention first time uses of such process to launch multiple attacking to ROD in the opposite direction using victim's retinal image signal with images or videos with some text words in projecting database from victim's side (TP's side) to cause response or reaction from ROD. Once ROD makes some responses or reactions which the victim (TP) can not make, the programmable logical controller (PLC) will make judgement and identification quickly. How to say, the art of war is called to turn somebody’ trick against himself or to beat somebody at his own game, because the automatic machine in this invention can change the passive harassment to victim into revealing the identity of victim and further more revealing all kinds of status of ROD himself/herself using device of V2K and RNM, although the ROD stay at a remote place.

Third, the special algorithm running in the CPU in a programmable logical controller (PLC) uses the automatic control curve to identify the tested person is a victim, then can identify the all status of a remote operator using device of V2K and RNM.

Summarize the above three steps, the invention is the world first using Automatic Control Technology—Programmable Logical Controller (PLC) to expose the huge amount abuse of V2K and RNM technology, it is only one countermeasure between Automatic Control Technology and V2K & RNM Technology at present, it is not a battle between human beings.

Different from human brain, PLC does not consider mistakes of electronic signals which a human being inputs by pressing buttons on a touchscreen by a person, because the special software in PLC choose a fluctuating trend which approaches correct goals finally, whereas human brain always consider choosing which one is correct or which one is wrong only, so this job can not be instead of human brain. The invention does not exist mind process.

PLC is different from ordinary computer, PLC have special CPU which can scan 1000 instructions within 100 ms in average, all kind of Timer (T0-T255) which are convenient for timing any length of time, all kinds of variable memories such as VB store Bit, VW store Word, WD store double words, all kinds of bit memory M 0.0-M 31.7, special memory SM, all kinds of Counters (C0-C255) and high speed counter HC0-HC5 and so on, so PLC can completes task which ordinary computer can not complete.

Furthermore, the invention has a very advanced automatic guide system, when a person chooses thousand pictures or videos from projector's classified database and press answer buttons on a touchscreen, the special PLC algorithm will show green light CORRECT or red light WRONG in the touchscreen within 0.01 second, so the guide system will let the tested person (TP) reach the final identification goal very fast.

At last the automatic machine can automatically print a certificate of victim to a person to identify him/her is a victim which can provide reference to medical department. Meanwhile, the automatic machine can also automatically print some document about remote attacker's or remote operator's all kinds of status which can submit to department of justice for reference.

The invention is an unique combination of existing technologies in the world, using an unique software which the inventor programs, generates an unique effect in the world in a field where on one has ever involved, so no doubt, it is a real invention.

An object of the present invention is to provide an automatic machine can identify both the victims of technology abusing and those remote operator's status using devices of V2K and RNM around world. This is the first set invention of identification combined with polygraph technology, projecting image technology, special automatic technology (PLC) and human-machine interface technology around world which can automatically generate identification results, except checking polygraph meter with human, all other parts of the system achieves automation.

The principle of the invention utilizes the remote operator can read retinal image signals which later transferred into image on a remote device which operator is using according these above US patents description, so the operator can see what a victim see via

Remote Neural Monitoring technology. The invention asks a victim to watch or only a glance some images or videos with some classified psychological attacking words against remote operator to send these retinal image signals to a remote operator and attack a remote operator directly to cause the remote operator classified psychological response which the victim can feel or sense according V2K feedback signal technology describing in these above US patents description. Then asking the victim to answer designed question on a touch screen with pressing buttons YES or NO,

American eye doctor with US PhD Ms. Diana Chao, M.D. explain very clearly that retinal image signal formed by some lights into retina is not mind process.

when a person glance or watch on something forming a retinal image signal only in a microsecond (equal to one millionth of a second) then transfers to remote device of V2K and RNM with the speed of electromagnetic wave transmission, these is not any mind process in the procedure.

The every image and video from projector database contains some per-designs text words to attack remote operators using device of V2K & RNM technology, so the victim's (TP's) retinal image with such text words only cause those remote operator's psychology reaction, but cannot cause TP's psychology reaction.

So the system is a kind of automatic machine inputting raw materials and outputting products as following simple diagram presentation, the retinal image signal forming like camera lens imaging function, so there is not any mind processing. Refer FIG. 11 i.

### Abbreviation in the Invention

V2K: Voice to Skull

RNM: remote neural monitoring technology

ROD: remote operators using devices of V2K and RNM technology

ISA: an identification system administrator

TP: a tested person who originally claims him/her as a victim

PLC: programmable logical controller

PM: polygraph meter (computerized digital)

TS: touch screen

Six Steps for Identification:

Step 1. Ask a TP who claims himself/herself as a victim to use of RODs reading his/her retinal cortex image signals and auditory neural signals in their cerebral cortex being monitored remotely to watch some images or videos with some words in databases in a projector to sent retinal cortex image signals to some remote operator using devices of V2K and RNM, (an American eye doctor with US PhD Ms. Diana Chao, M.D. explain very clearly that retinal image signal formed by some lights into retina is not mind process). This phase may take about five seconds.

Step 2. Then these retinal image signals will be shown on those devices as image with words only attacking ROD will cause those ROD's psychological response and at same time the TP can feel or sense those ROD's psychological responses according V2K feedback signal technology on TP's auditory cortex. The technology comes from US patents and China patents above and the base is microwave hearing effect or call Frey effect. This phase may take three seconds.

Step 3. Ask the TP to answer pre-designed questions which shows a touch screen by pressing buttons YES or NO on a touch screen according which psychological response from RODs. The YES or NO button on a touchscreen via M (Bit memory) communicating with M (Bit memory) in a PLC, via variable memory VW make a judgement and response within 0.01 second via M (Bit memory) to show green light “CORRECT” or red light “WRONG” on the touchscreen. The timer (T) in PLC keeps these showings ‘CORRECT’ or ‘WRONG’ on for two sends on the touchscreen. Then the TP see the small green light “CORRECT” or small red light “WRONG” on the touchscreen. The base of technologies come from CPU in a programmable logical controller make judgement with automatic control algorithm and basic human-machine interface asking person to make human-machine interaction. So from phase 1 to phase 3 only takes 10 seconds.

When step 4 finish, return to step 1 into a circulation with a high speed advanced automatic identifying guide system responds the tested person (TP) pressing YES or NO button only within 10 ms indicating CORRECT with small green light or WRONG with small red light. According automatic control decay curve (PID control regulate curve to prove the identification is accurate via a number of times of successful identification being divided by the number times of failure identification with sending multiple times of retinal cortex image signals to RODs and getting multiple time of psychological responses from RODs. Then the tested person (TP) can choose next image or next video in projecting database. So the automatic identifying machine can guide tested person (TP) quickly to reach identifying result and nearly does not need tested person (TP) to make some judgment, more than 90% judgments makes by the automatic identify machine. As the error curve approaches the X axis gradually. The base of theory come from automatic control theory.

There are a, b, c, d, e, f and g seven databases total, complete one of seven databases taking about 30 times to 50 times reverse attacking to ROD, one time of attacking only takes 10 seconds, so the invention can complete one database within 300 seconds to 500 seconds to reach a identifying result.

Step 5. When these identification finish database a, b and e successfully, the TP has been identified as a victim of abuse of V2K and RNM technology, the automatic machine will show the TP is a victim on the touchscreen and print a certificate of victim automatically.

Step 6. When TP finishes one of seven classified picture or video (a,b,c,d,e,f,g) projecting database identification, ISA connects PM to TP to confirm TP answering all questions on the touchscreen is honest. In case of TP complete this phase is not honest, ISA had to ask TP to repeat this identification phase.

Step 7. The information of victim and information of ROD will be stored into a victim database and a ROD database in standardized format; these information in the database can be real time update by a content server; and there is remote access to users over a network and transmitting the message to users over the computer network when information update.

However, the method only ask the TP to choose some contents in some database to send retinal cortex image signals to RODs when TP see some images or videos with some words in some database in a projector, in this phase, there is not TP's mind process. The next phase, TP answers very simple fixed questions on touch screen according the TP feels or senses which psychological response from RODs according V2K technology signal feedback on TP's auditory cortex, meanwhile the automatic guide system shows CORRECT with small green light or WRONG with small red light on the touchscreen on every pressing YES or NO button,

here is nearly not TP's mind process also, because microwave hearing effect or Frey effect does not belong mind process.

According theory of psychoanalysis: the psychological response between an active psychological attacker and a psychological attacked person passively have completely different characteristics, so these different psychological responses caused by selective classified psychological attacks can be used both to identify a victim (TP) and those remote operators using device (RODs) of RNM and V2K via satellite, even the victim (TP) is in USA, but the remote operator is in China.

Additional advantages and features of the invention will become apparent from the description which follows, and may be realized by means of the instrumentalities and combinations particular point out in the appended claims.

Still further objects and advantages will become apparent from a consideration of the ensuing description and drawings. These and other objectives, features, and

advantages of the present invention will become apparent from the following detailed description, the accompanying drawings, and the appended claims.

### BRIEF DESCRIPTION OF THE DRAWINGS

FIG. 1A is a schematic diagram showing Automatic Identification Machine Overall Layout; FIG. 1B is an Operating Electric Box; FIG. 1C is an Operating Electric Box Parts List Table; FIG. 1D is a Touchscreen Front Side and Touchscreen Back Side; FIG. 1E is a Programmable Logic Controller; FIG. 1F is a RS 232 Cable connected Programmable Logic Controller to Touchscreen; FIG. 1G is a Polygraph Meter; FIG. 1 H a Databases in Image and Video for Projector System; FIG. 1 I is the Automatic Identifying Machine Input and Output Schematic Diagram; and FIG. 1 J have Document Database with automatic real time update, Printing System, and Access to users over a network with transmitting the message to all user over network when information update.

FIG. 2A to FIG. 2F illustrates a first set of flowcharts for identify a victim according to the present invention.

FIG. 3A to FIG. 3E illustrates a second set of flowcharts for identify all kinds status of remote operator or attacker (ROD's) according to the present invention.

FIG. 4 illustrates an identification decay curve.

FIG. 5 illustrates examples of disgusting things with TP's retinal image signals with a picture involving some words attacking ROD directly.

FIG. 6 illustrates examples of funny stories with TP's retinal image signals with a picture involving some words attacking ROD directly.

FIG. 7 illustrates examples of political and religious attack with TP's retinal image signals with a picture involving some words attacking ROD directly.

FIG. 8 illustrates examples of nuclear attack with TP's retinal image signals with a picture involving some words attacking ROD directly.

FIG. 9 illustrates samples identifying ROD's scope of language and dialect in which TP use a touchscreen which connects with a programmable logical controller and a projector image to achieve automatic identification.

FIG. 10 illustrates examples of identifying ROD knows what scientific and technical knowledge with TP's retinal image signals with a picture involving some words attacking ROD directly.

FIG. 11A illustrates how TP use a touchscreen which connects with a controller and a projector image to achieve automatic identification; and FIG. 11B illustrates how TP use a touchscreen which connects with a programmable logical controller and a projector image to achieve automatic identification.

FIG. 12A illustrates how TP use a touchscreen which connects with a programmable logical controller and a projector image to achieve automatic identification; and FIG. 12B illustrates how TP use a touchscreen which connects with a programmable logical controller and a projector image to achieve automatic identification.

FIG. 13 illustrates how TP use a touchscreen which connects with a programmable logical controller and a projector image to achieve automatic identification.

FIG. 14 illustrates how TP use a touchscreen which connects with a programmable logical controller and a projector image to achieve automatic identification.

FIG. 15A illustrates a Special PLC algorithm for identifying disgusting response; FIG. 15B illustrates a Special PLC algorithm for identifying funny response; FIG. 15C illustrates a Special PLC algorithm for identifying political ideology and religious belief response; FIG. 15D illustrates a Special PLC algorithm for identifying ROD″s nationality and race; and FIG. 15E illustrates a Special PLC algorithm for identifying a tested person who is a victim of abuse technology of V2K and RNM and can identify ROD belong which organization. Note: FIG. 15A, FIG. 15B, FIG. 15C, FIG. 15D, FIG. 15E are only PLC algorithm suitable described general PLC algorithm, they do not belong to any specific PLC company or any specific PLC manufacturer's software programming.

FIG. 16 illustrates an Automatic Identification Machine including tested person VS Device of V2K and Device of Remote Neural Monitor including remote operator.

### DETAILED DESCRIPTION OF THE PREFERRED EMBODIMENT

V2K: What is ‘Voice to Skull’? . . . Voice to skull is the transmission of voice, or any other audible or subliminal sound, directly into the hearing sense of a victim. So the invention is based on technologies about of RNM and V2K technology around the world. The painful experience described by victims is consistent with the effect of these technologies in relation to V2K which were described in US patents and China patent.

The invention is based on these V2K and RNM device operated by human beings and these operators show themselves emotions via microphones and earphones when they are using these devises to harass a victims. On the other hand, the victim can feel and sense operator's emotions via V2K technology effect on their auditory cortex. This emotional interaction between victim and operators is interactive when these V2K and RNM devices are running.

The reason to confirm V2K and RNM device operated by human beings is human brain can forget something, because operators frequently forget somethings related victim's history, but victims do not forget these things in himself or herself history. On the other hand, these computer memory and robot memory with artificial intelligent never lost data when programmer programs data into their memory, so only human has memory error, but computer's CPU never has memory data read and write error. So the invention can analyze operator's human emotion which a victim can feel and sense to identify these voice to skull is not victim's auditory hallucination, but they are emotional response from those remote operators. Because there are obvious differences between victim's emotion reaction and those emotions response belong to remote operators using devices of V2K and RNM, so if using the invention method, you can find these differences clearly.

### STATEMENT OF GOVERNMENT INTEREST

The invention can help victims to confirm their grieves and get justice, on the other hand, it can help law enforcement like FBI to find out the fact of cases, as this remote technology is global and transnational crime, so if all kinds of victims enter United States in some related cases, FBI can use the invention as a tool to analysis and identify who are remote operators (or call them remote attacker), then these operators' (or call them remote attacker) nationality and race; religious belief and political belief, scale of language and dialect, scale of scientific knowledge, even identify these attacker or operator belong to which organization.

For example, FBI investigated the case of Washington D.C. navy yard gun case, CNN reported on Sep. 26, 2013, the gunman suffered “Low frequency attacks” and that attack drove him to kill. If similar cases happen, the invention can help FBI to investigate.

Declaration: In this invention, all political, religious attacks or attack a country are human attacks with viewing videos, pictures & other materials in computer databases only are retinal image signals with some words attacking remote operators using device (RODs) to cause remote operators using device (RODs) psychological response in non-public site, there is neither any verbal attack nor behavioral attack. So do not cause insulting anyone.

Description of the Automatic Machine of the Present Invention:

The invention first time apply principle of automatic control attenuation cure to gradually approaching final correct direction on human multiple instinct selections, regardless the human's single selection is correct or wrong, because the invention has an advanced automatic guide system to indicate every correct answer and every wrong answer which can guide the tested person (TP) to get final identification goal, just requires this tested person (TP) is an evolved human being, the invention can reach final correct result, so such invention will be the first one around the world.

So the invention uses programmable logical controller as main component:

1. Use some intermediate registers M in PLC as communicating component connected PLC and a touchscreen, when there is a pressing button signal on touchscreen, CPU in PLC response within 10 ms (0.01 second) to send judgement of input signal is correct or wrong and meanwhile make reaction let the touchscreen show green light “CORRECT” or red light “WRONG”, the intermediate register M also make last instruction to make touchscreen show identifying result on the touchscreen.

2. Use some variable registers VR in the CPU of a PLC to do all kinds of judgment calculations, in this invention uses of VW.

3. Use some precise timers in the PLC to calculate time, the calculation accuracy reaches 10 milliseconds.

All these technical means insure the last identifications are correct.

The following is a well-known automatic control PID algorithm formula:

u₍ₖ₎=kₚ(err₍ₖ₎+kᵢΣerr₍ⱼ₎+k_{d}(err₍ₖ₎−err₍ₖ₋₁₎)),

Kp: scale parameter; Ki: integration parameter; Kd: differential parameter.

The invention utilizes a victim's (TP's) intentional selective and classified retinal image signals with some words attacking remote operators using device (RODs) of V2K and RNM to cause remote operators using device (RODs) classified psychological response or classified emotional responses which victim (TP) can feel and sense according V2K technology effect on victim's (TP)'s auditory cortex. So, there is nearly not mind process.

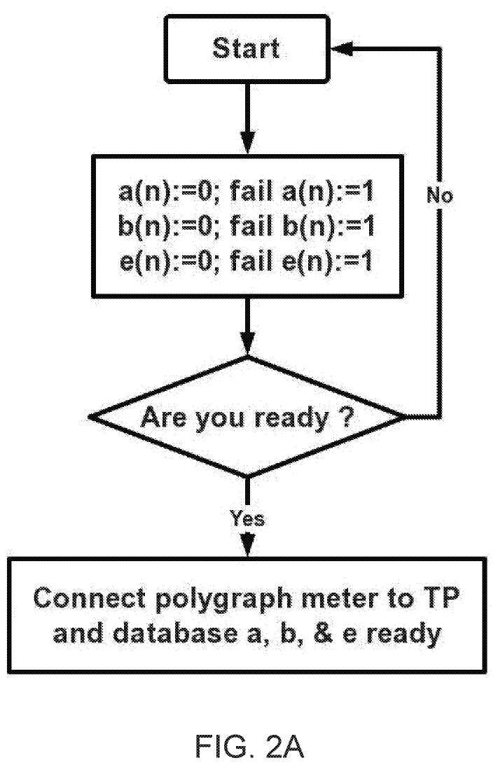

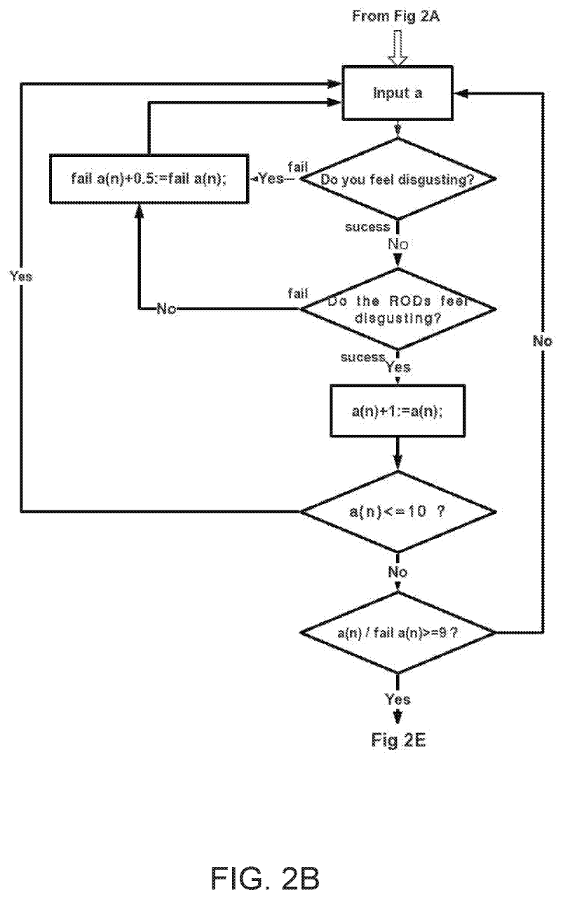

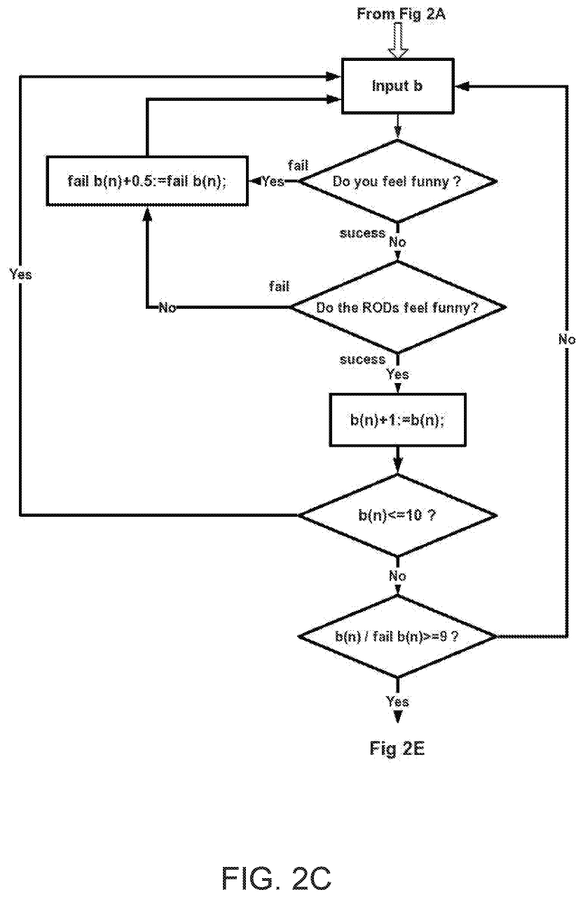

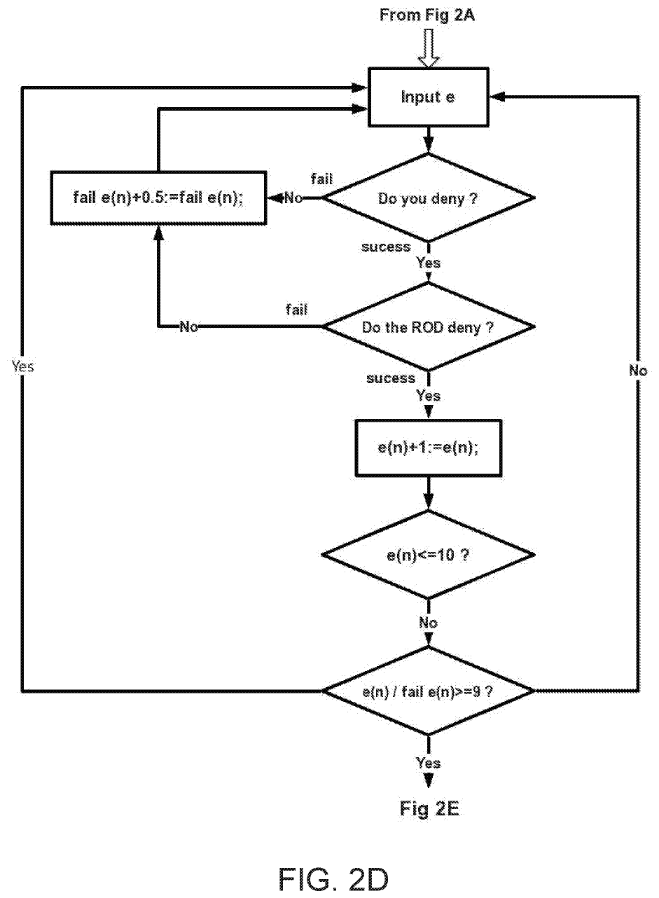

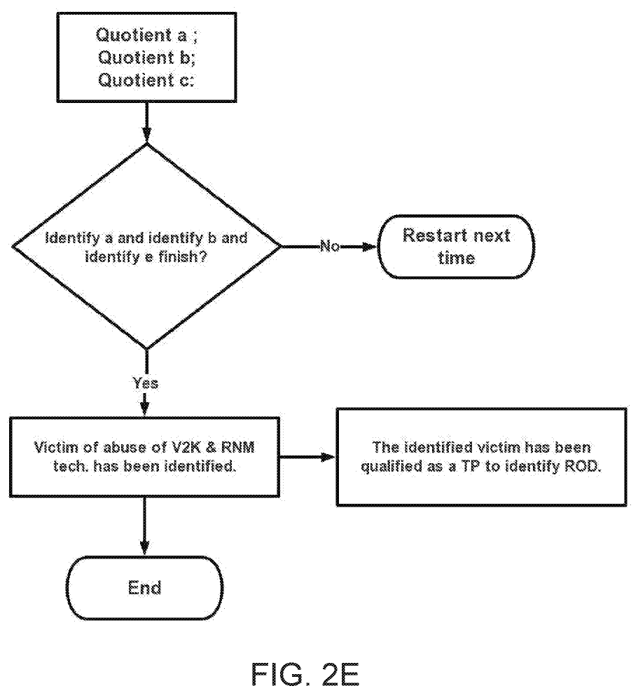

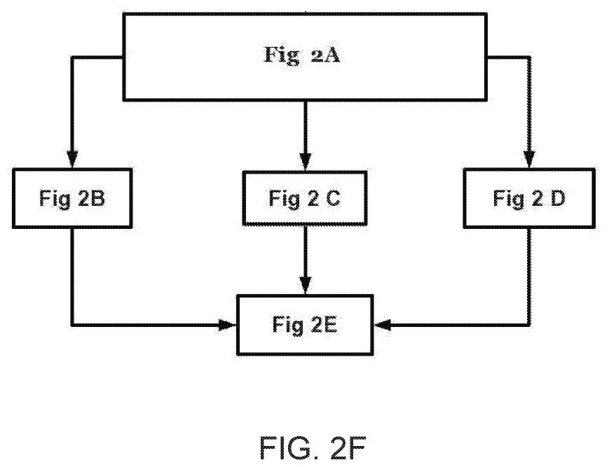

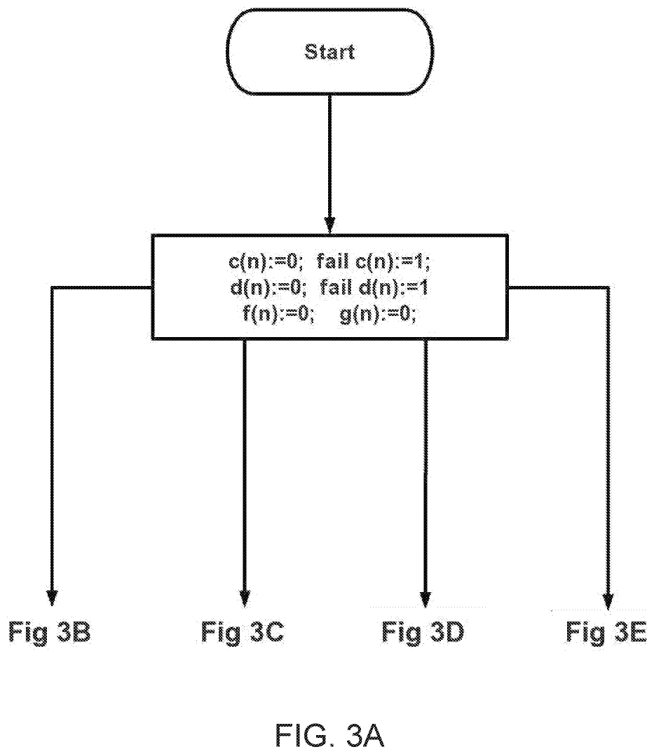

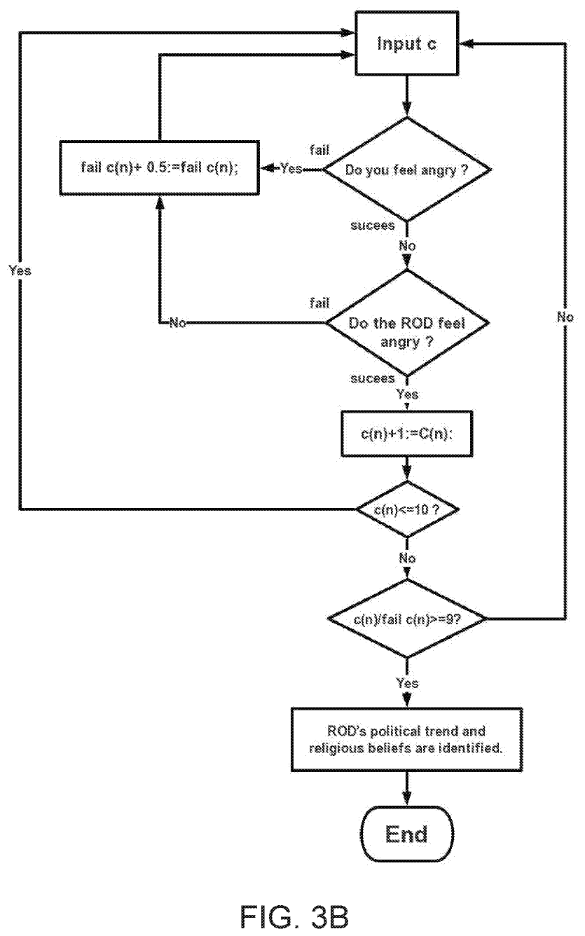

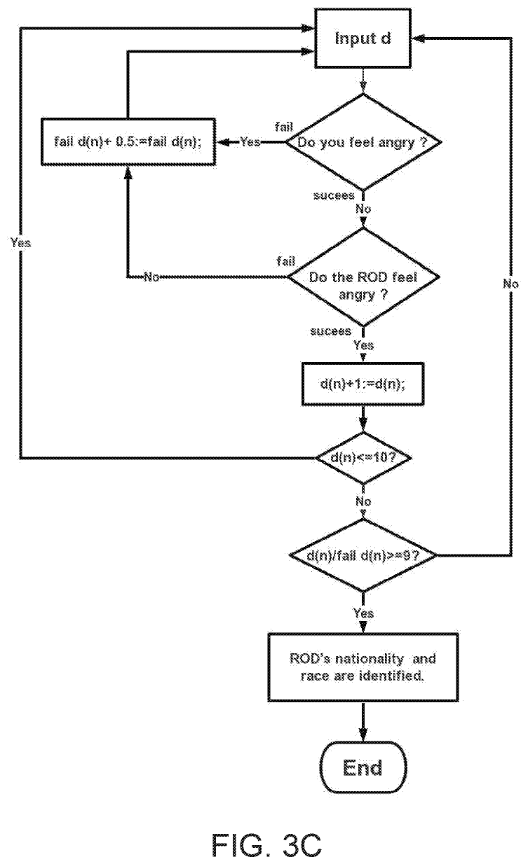

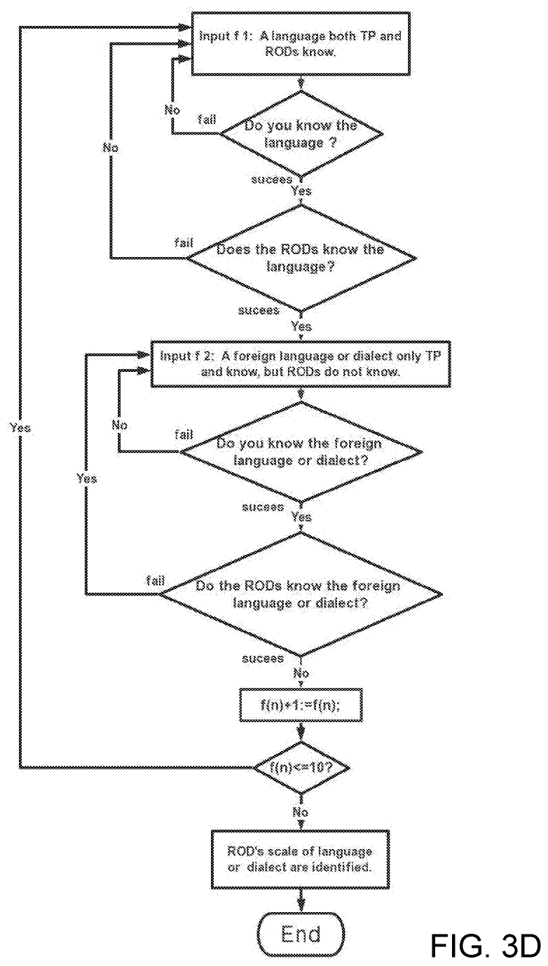

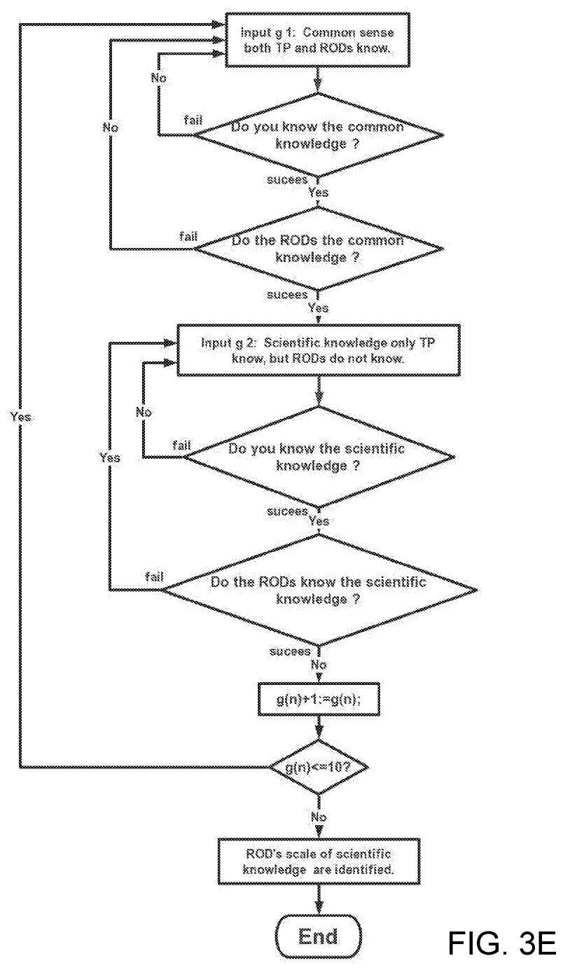

Then the invention uses of relatively comparing methods, designed procedures in flowcharts classify emotional responses between the victim's (TP's) and remote operators' (ROD's), designed procedures in flowchart can find out there are significant differences between victim's (TP's) and remote operators' (ROD's) with judgement diamond frames. Use of these difference, a programmable logical controller (PLC) can identify both a victim (TP) and classified identify all kinds status of remote operators (ROD) using devices of V2K and RNM by checking flowcharts FIG. 2 A to FIG. 2F and FIG. 3A to FIG. 3E automatically.

These classified identities including remote operator's (ROD's) nationality, race, political trend, religious belief, organization, scope of language and dialect, scope of scientific knowledge.

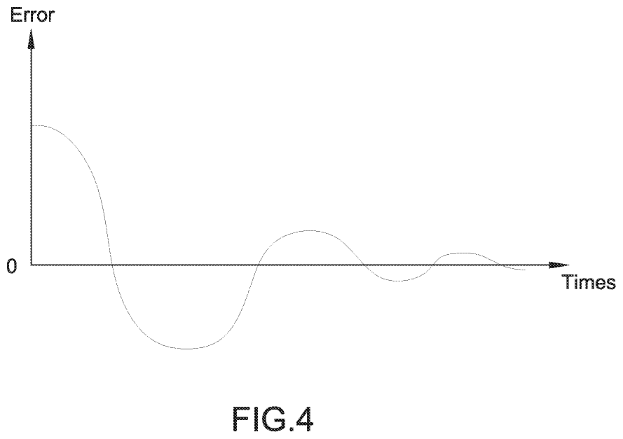

When identifying begin, a identifying curve indicates identifying error changes gradually from large to small and approach to accurate identification at last, the principle is similar with automatic control decay curve, which is shown in FIG. 4.

Such similar curve is widely used for PID controller in automatic control technology which is used to control like temperature and pressure, but the invention uses it to identify who is a victim (TP) and to identify all kinds status of a ROD, mainly for identifying ROD.

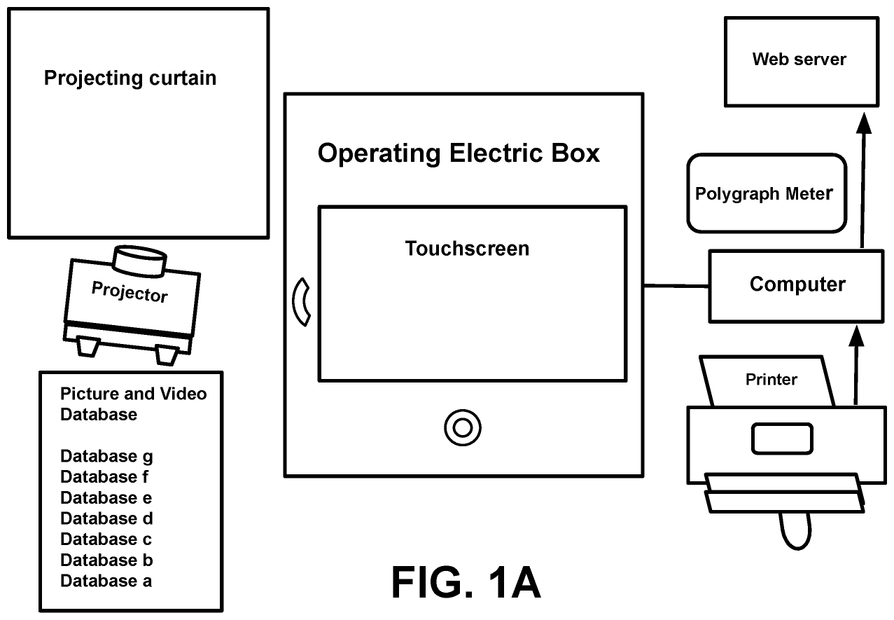

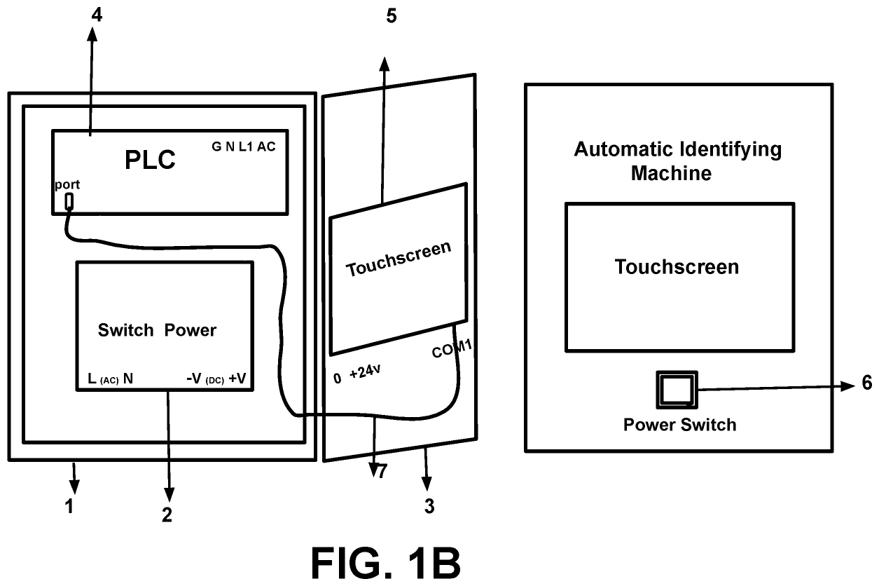

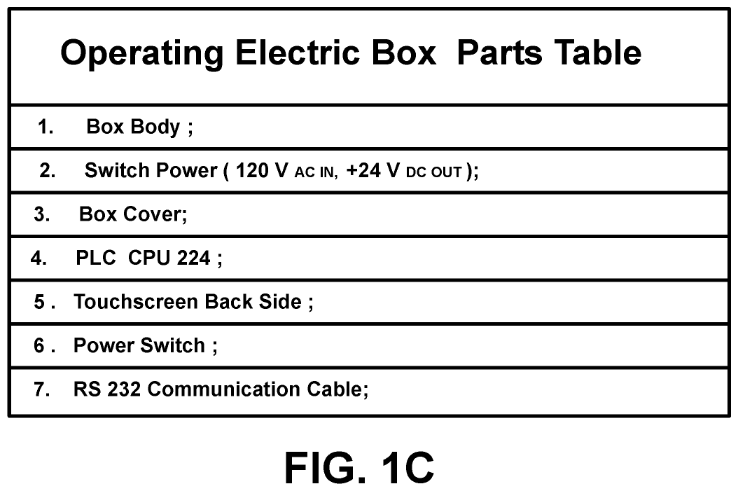

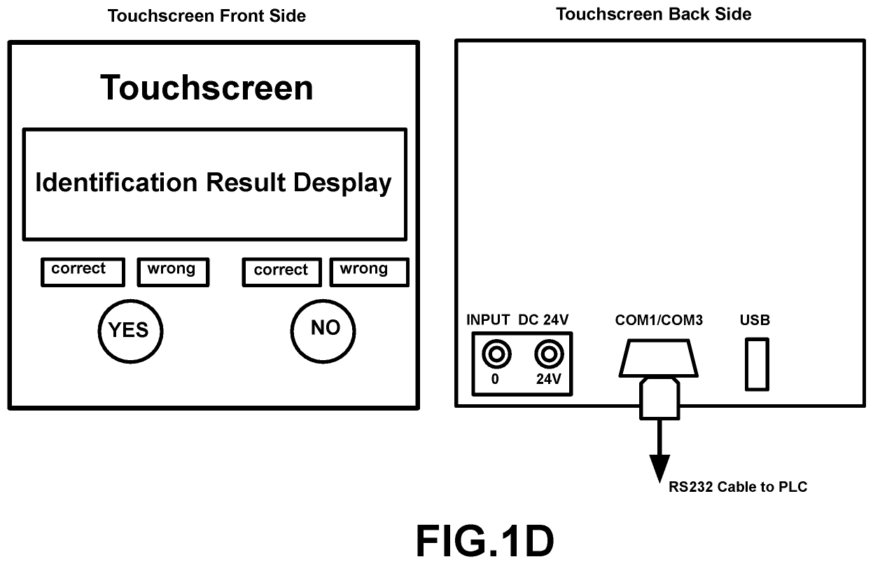

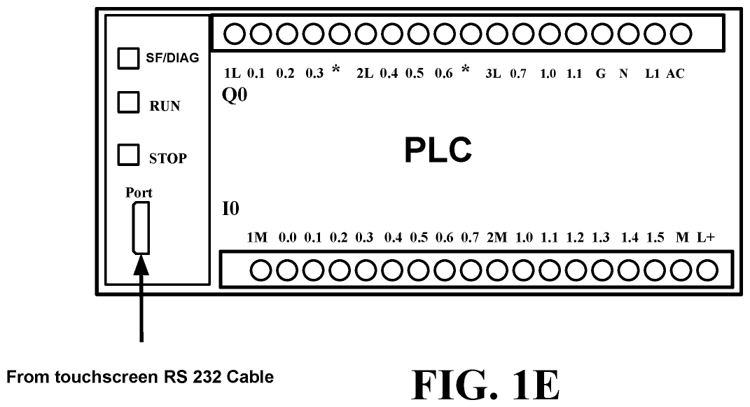

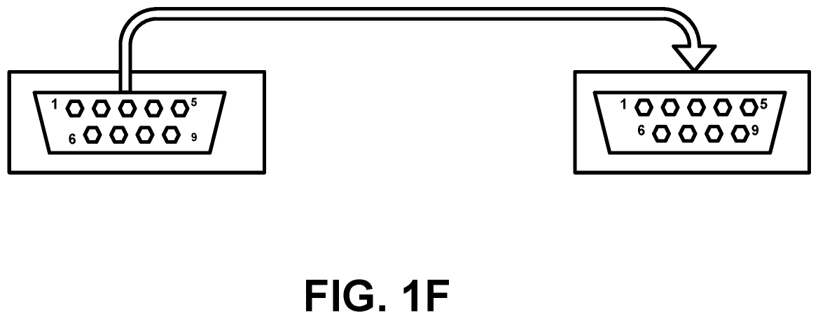

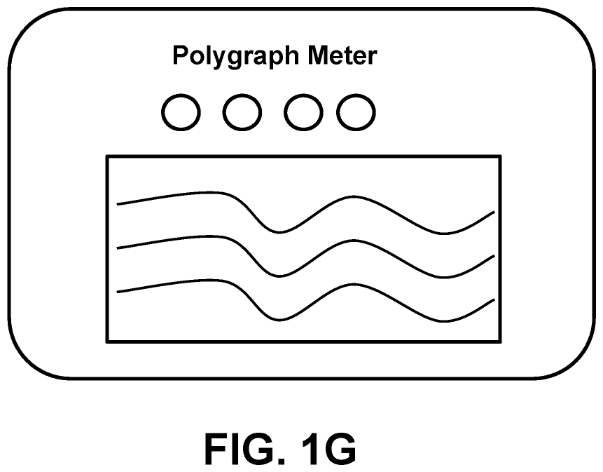

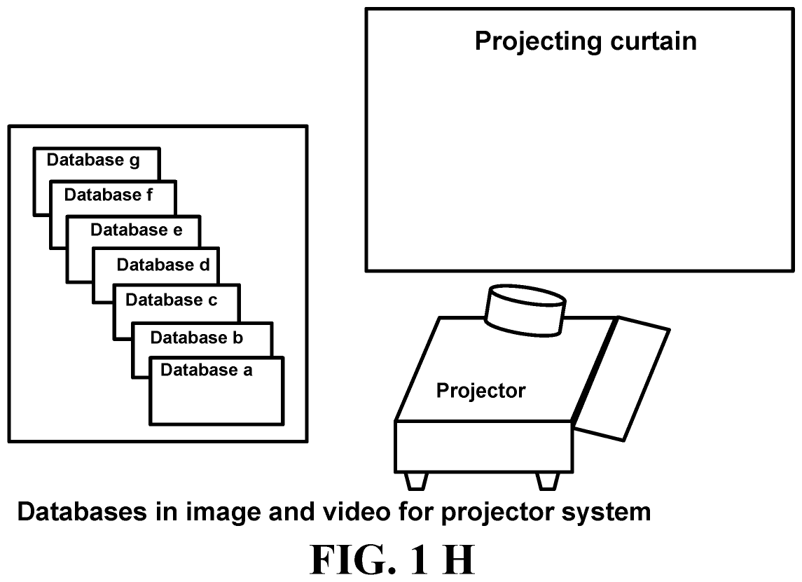

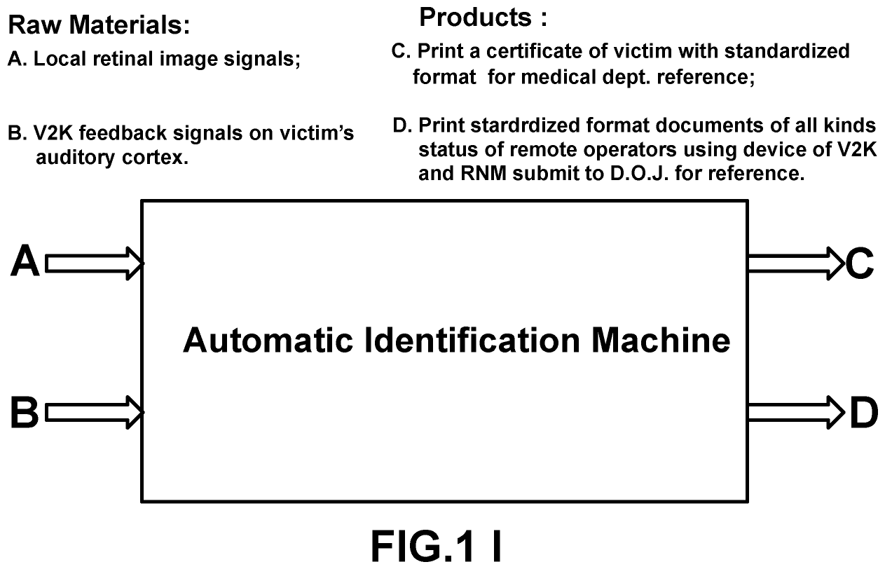

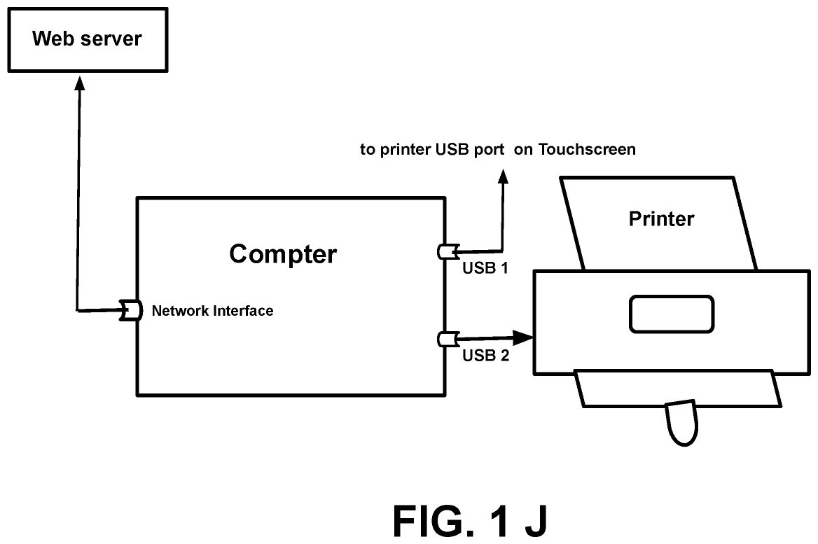

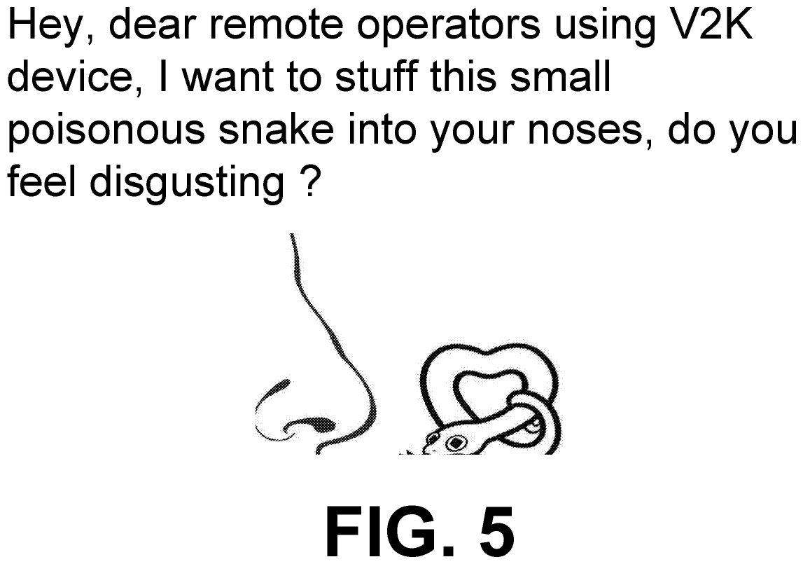

Referring to FIGS. 1-14, the present invention consists four hardware parts:

1 . . . polygraph Meter: (PM)

Polygraph meter is used to confirm the tested person's (TP's) answer is true.

2. Operating Electric Box: there are a Programmable Logical Controller, a Touchscreen, a Switch Power, a Power Switch in the Box;

3. Projecting image and video system, there are a projector, a database for images and videos and projecting curtain.

4. Printing and storing system: there are a printer and a computer and there is an identified information database in the computer with an access of network for users.

Referring to FIGS. 1-14, the present invention consists two persons:

1. ISA: An Identification System Administrator.

An identification administrator (ISA) asks a tested person (TP) to choose some special pictures or videos with some words in some databases in a projecting system to send retinal image signals those remote operators (ROD)'s to cause those remote operator's, (ROD)'s psychological response via V2K and RNM device, but cannot cause tested person's (TP) same psychological reaction. Meanwhile, the tested person (TP) to answer some designed questions which shown on a touch screen with pressing designed buttons YES or NO on a touch screen according diamond frames in flowcharts in a computer what remote operators' (ROD's) psychological response which the tested person (TP) can feel or sense according V2K technology on TP's auditory cortex. ISA asks the tested person (TP) answer all questions on a touchscreen honestly before test begin, then uses the polygraph meter to confirm TP is honest to answer questions.

2. Tested Person (TP)

Tested person (TP) should claim himself/herself is a victim of abusing V2K and RNM technology. Tested person answer questions which can be analyzed and identify who is the remote operator (ROD) using V2K and RNM device, furthermore the invention may analyze and identify a remote operator's (ROD's) nationality, race, political belief and religious belief, scale of language and dialect, scale of scientific knowledge and which organization by the designed procedures in a computer and a programmable logical controller.

Of course, the qualified tested person (TP) should be emotional stable and rational thinking, as well as cooperative with identification system administrator (ISA).

A projector database contains seven classified databases a; b; c; d; e; f and g which contain classified pictures and videos with some words attacking remote operators using device (RODs) directly. Every classified database can let tested person (TP) choose contents to send some classified retinal image signals with some words attacking remote operators using device (RODs) to cause ROD's classified psychological responses.

This the ROD's classified psychological responses can be used to identify who is victim (TP) and who is remote operator (ROD) by operating programs in flowcharts in a programmable logical controller.

Of course, the classified contents in database can be updated and increased according different requirements in a projector used in identifying process.

A programmable logical controller (PLC): A programmable logical controller manages special algorithm when automatic system is running. It also contains twelve counters in flowcharts, flowcharts is a programmed software, among them, seven counters are successful counters which record every time successful identification and accumulate the times of successful identification: a(n), b(n), c(n), d(n), e(n), f(n) and g(n). Another five counters are failure counters which record every time failure identification and accumulate the times of identification: fail a(n), fail b(n), fail c(n), fail d(n) and fail e(n).

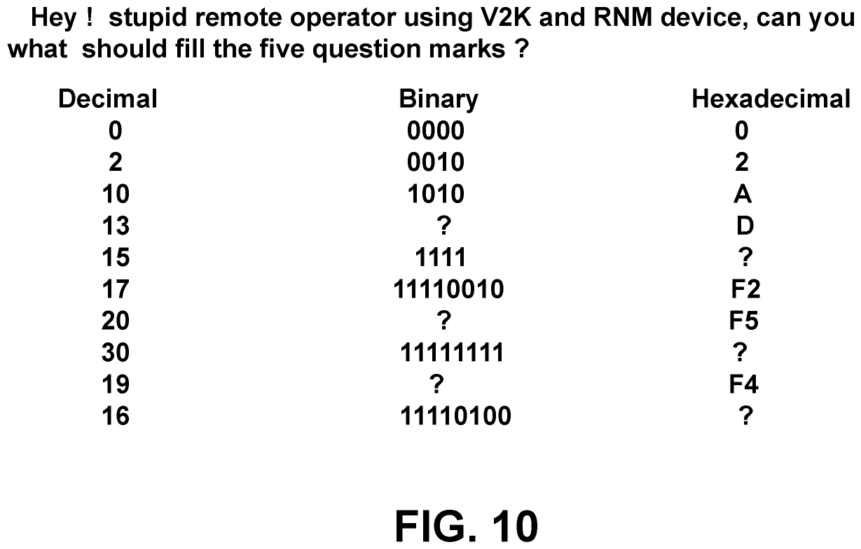

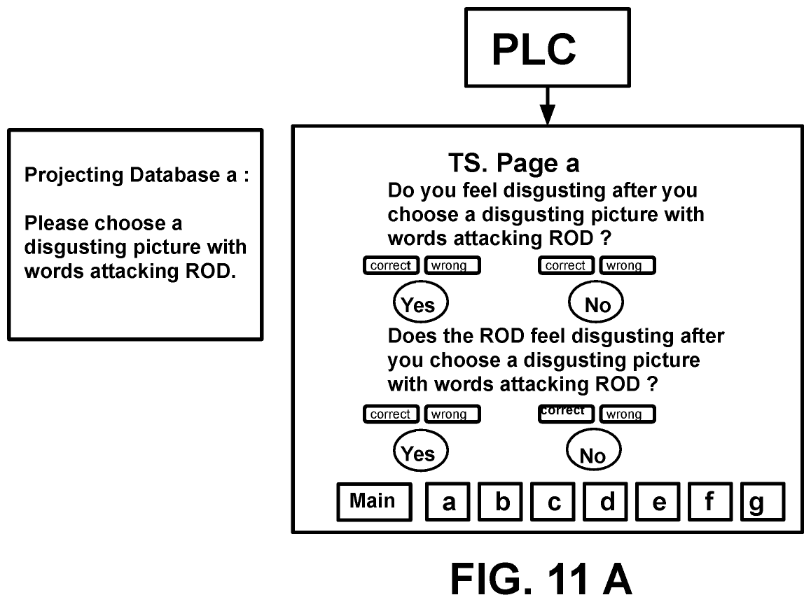

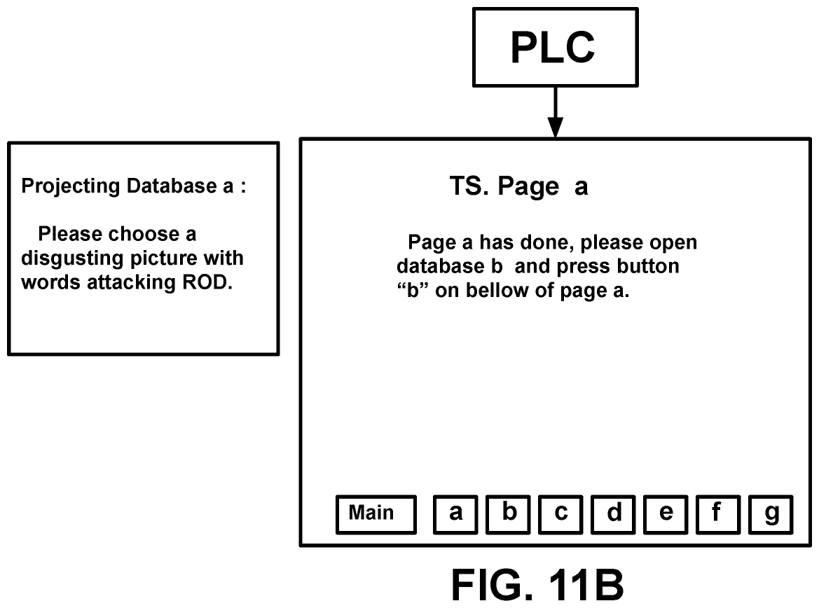

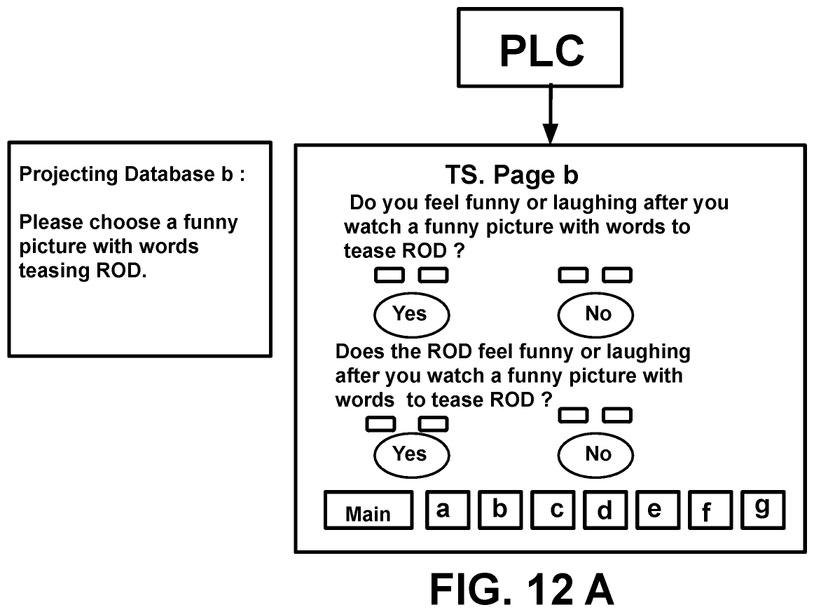

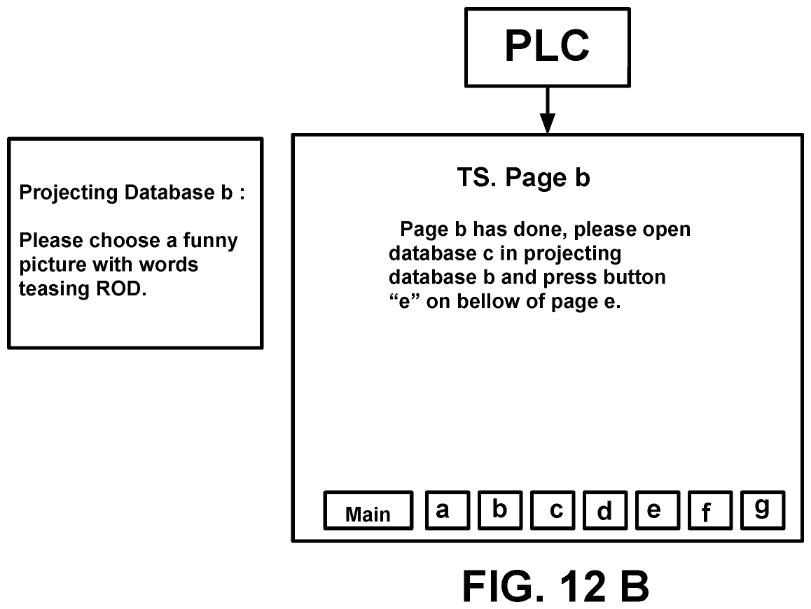

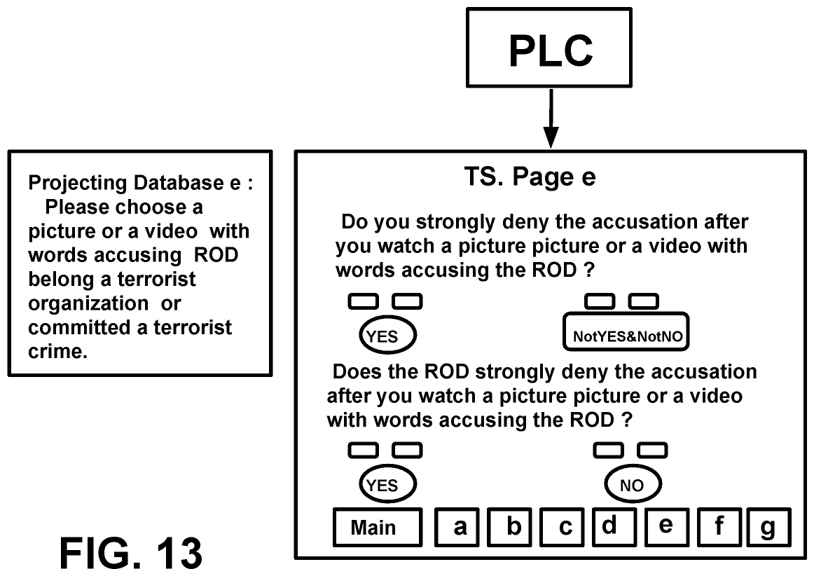

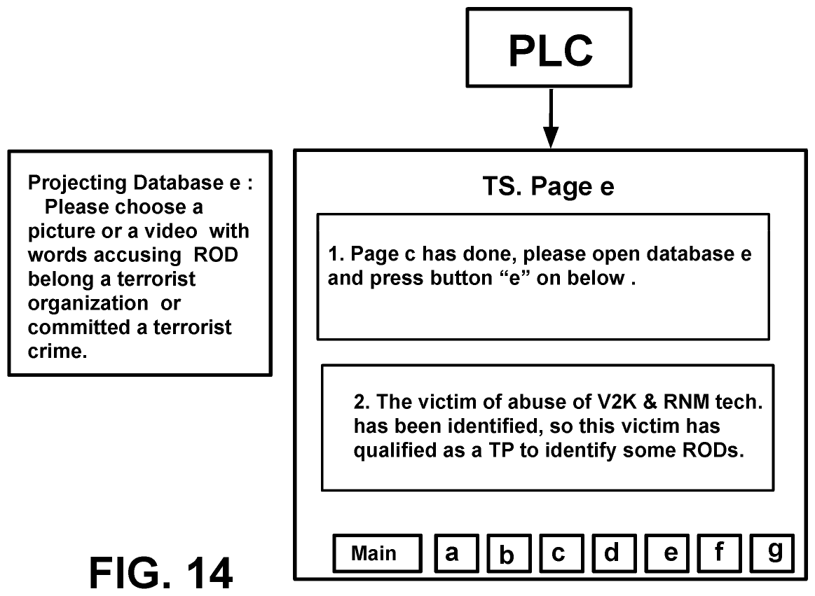

A touch screen (TS): the touch screen will have eight pages, except the main page FIG. 10, they are FIG. 11A; FIG. 11B; FIG. 12A; FIG. 12B; FIG. 13; FIG. 14; these Figs have been drawn out, other FIG. 3A to FIG. 3E are similar, so does not been drawn out. Touch screen operating pages represents one section of flowchart shows designed questions to TP, TP also answers questions with pressing “YES” or “NO” buttons on touch screen; meanwhile the touchscreen will show these answers with green light CORRECT or red light WRONG. Finally, the touch screen also shows result of identifications with English (or other languages) displaying window to TP and ISA. At same time the touch screen connected with programmable logical controller with some baud rate (how much digital bits transfer per second). All programs are running in background programmable logical controller.

Judgement contains in every diamond in flowcharts, TP answers questions in every diamond frame which shows on a touch screen.

Every diamond frame have two flow lines directions, a word “success” beside the line indicates one time of successful identification; a word “fail” beside the line indicates one time of failure identification.

There are two sets of flowcharts which are programmed software in a programmable logical controller.

The first set of flowcharts consist of FIG. 2A to FIG. 2F which are used to identify a victim (TP) of abuse technology of V2K and RNM, the input contents from database a, b and e; there are three successful counters: a(n), b(n) and e(n), there are also three fail counters: fail a(n), fail b(n) and fail e(n).

Among these fail counters, they represent any failure identification, fail a(n), fail b(n), fail e(n) in first set of flowcharts; but fail counters fail c(n) and fail d(n) in second set of flowcharts.

In the identifying process, the number in successful counters is divided by the number in fail counters, if the quotient larger or equal to 9, the identification has been completed. In case of need, the quotient can be larger than 9.

In order to avoiding the divisor (denominator of the score) is zero, we assign an initial value 1 to every fail counter. In the fractional number in a(n), b(n), c(n), d(n) and e(n) are numerator, but the number in fail a(n), fail b(n), fail c(n), fail d(n) and fail e(n) are denominator.

The second set of flowcharts consist of FIG. 3A to FIG. 3E, which are used to identify those remote operators' or attackers' (ROD's) all kinds status, who are using devices of V2K & RNM, the input contents from database c, d, f and g.

There are also two successful counters: c(n) and d(n), two fail counters: fail c(n) and fail d(n), but there are not fail counter fail f (n) and fail g(n).

Each flowchart input contents according type and characteristics from contents database in a computer, ISA asks TP to choose some suitable contents form the database to send a retinal image signals to ROD, then TP answer questions which show on a touch screen in diamond frame in flowcharts about what kind of psychological response from ROD which TP can feel and sense via V2K technology on TP's auditory cortex. Seven category of input contents are in seven classified databases.

Every projecting system database stores different content like following, but these contents in different databases inputs classified content only the method put into using. For examples, database of category d can input twenty five names of countries and more different races, because it was said there were twenty five countries own the technology of remote neutral monitoring and voice to skull or relevant equipment. So ISA can store these names of twenty five countries and their races in database d of a computer when the method in operating.

After a TP answers the questions with pressing button YES or NO, the touchscreen will show small green light CORRECT or small red light WRONG at once and keep the lights on for two seconds, which can guide TP to get final identification fast.

Seven category of input contents in seven classified databases, each database stores different content like following, but these contents in different databases store classified contents only the method put into running.

The present invention designed that if a TP passes database a, database b and database e identifications, the TP has been identified as a victim of abuse of V2K and RNM technology, the automatic machine will print a certificate of victim to this TP.

Then the invention regards the TP as a qualified tested person to identify ROD's all kind status further as following:

1. By the help of the qualified TP, complete database c to identify ROD's political trend and religious belief

2. By the help of the qualified TP, complete database d to identify ROD's nationality and race.

3. By the help of the qualified TP, complete database f to identify ROD's scope of language and dialects.

4. By the help of the qualified TP, complete database g to identify ROD's scope of scientific knowledge.

Of course, 1, 2, 3 and 4 identifications can also strengthen the accuracy of identification of a victim, because all these features in 1, 2, 3 and 4 identifications belong ROD, they do not belong the victim or call TP any more.

Category A:

The category a database contains some universal disgusting pictures and videos with some words which are specifically only causing remote operator's (ROD's) using devices of V2K and RNM disgusting psychological response, but they will not cause a tested person's (TP) disgusting psychological reaction, because these words in pictures and videos only attacking ROD directly, but not any words attacking the TP.

After a TP has chosen a kind of disgusting picture and videos with some words attacking ROD, the TP has sent his/her retinal image signals to ROD. Then ROD must make psychological response when ROD see these disgusting picture and videos with some words or sentences attacking him/her on ROD's device. There is nearly not mind process.

After TP feel or sense what psychological response from ROD, TP answer question in diamond frame in flowchart which shows a touchscreen by pressing buttons “YES” or “NO”, every time TP press a button, a small green light show “CORRECT” or red light show “WRONG” on touchscreen which the automatic system guides TP gradually to the final accurate identification fast.

After category identification, an identification system administrator (ISA) connects PM to TP and then asks TP one question: ‘Did you answer these question on this touchscreen honestly?”, TP answer; “Yes, I did”. ISA checks PM to confirm TP's answer is true, then finish identification. In case of TP complete this phase is not honest, ISA had to ask TP to repeat this identification phase.

According automatic control theory, multiple TP's disgusting retinal image signals psychological attack cycles will generate precise identification: the disgusting psychological response comes from ROD, but does not belong to TP.

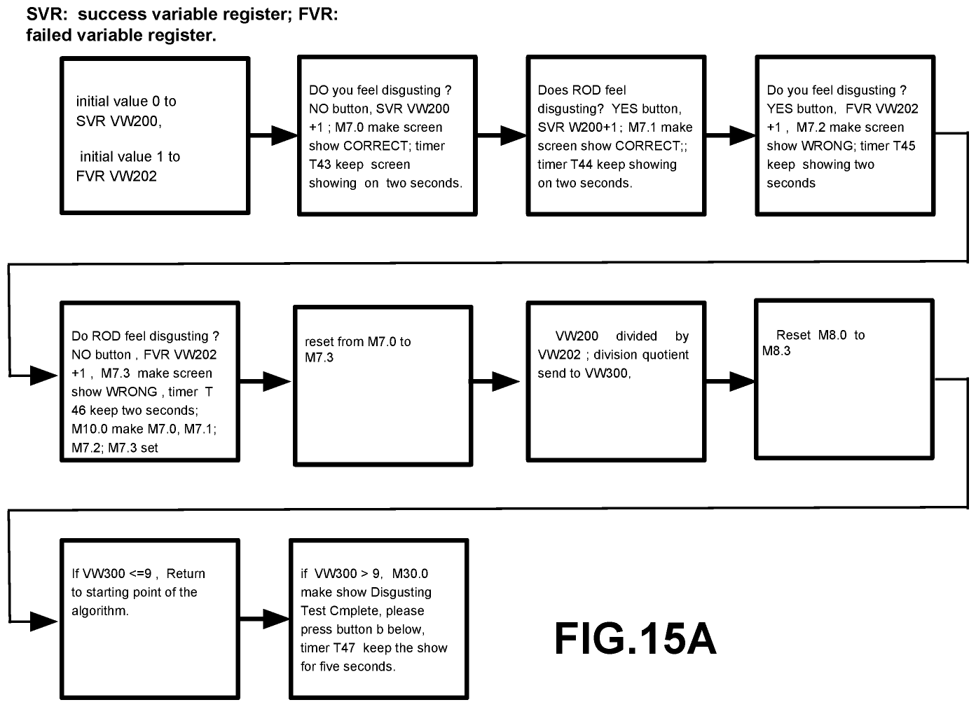

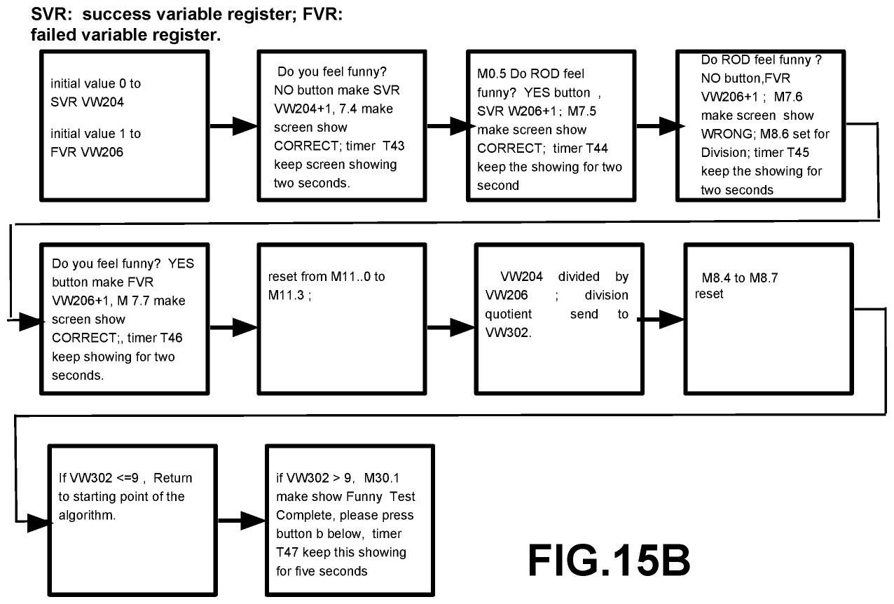

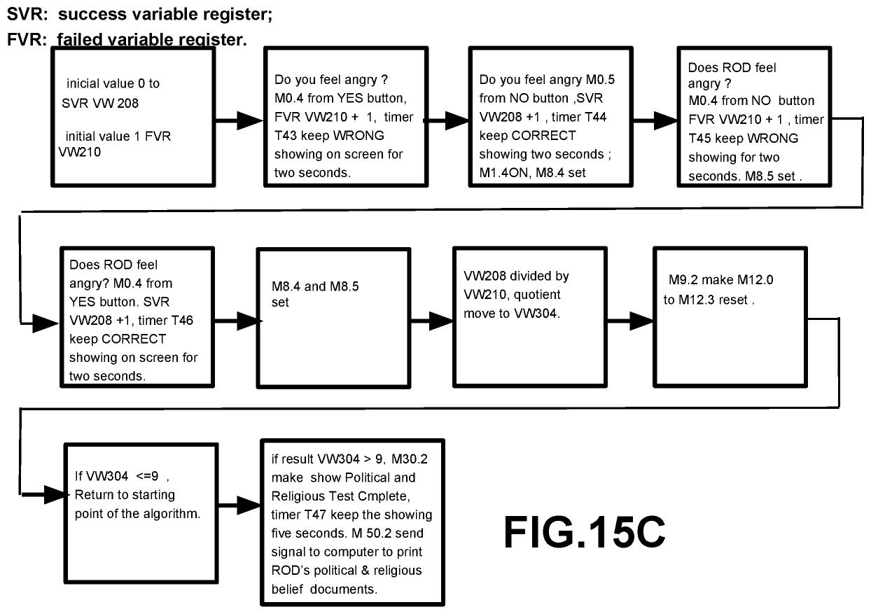

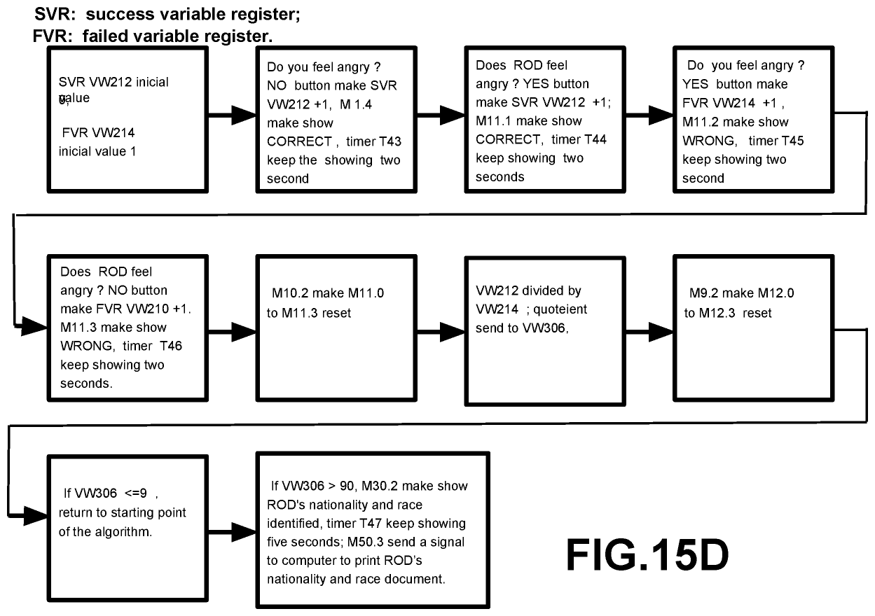

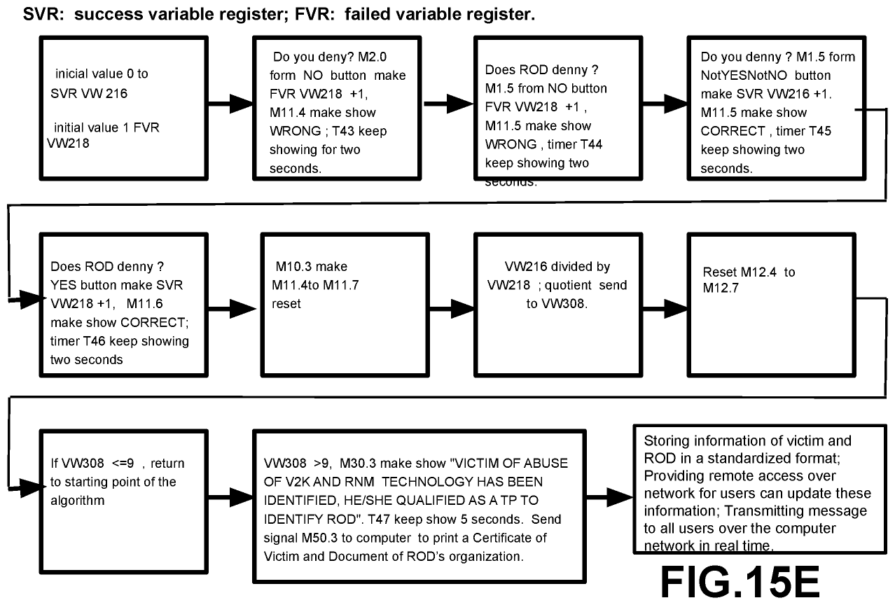

Special PLC algorithm for identifying disgusting thing reaction V2K signal refer FIG. 15 A.

Disgusting Images or Videos Example List:

| | |
|---|---|
| 1. | Picture or video of a pile of roundworms with |
| | words or sentence :.” Hey, |
| | these ugly roundworms only for your operators using |
| | device of V2K and RNM as breakfast noodle.” |
| 2. | Picture or video of a piece of necrotic liver from a |
| | liver cancer patient with words or sentence :” Hey, |
| | the ugly cancer liver only for your operators using |
| | device of V2K and RNM as main dish in your dinner”. |
| 3. | Picture or video of a piece of leprosy skin from |
| | a leprosy patient with words or sentence :” Hey, |
| | the ugly skin only for your operators using device of V2K |
| | and RNM as your dessert”. |
| 4. | Video of some blood of advanced AIDS patient |
| | with words or sentence :” Hey, these disgusting |
| | blood only for your operators using device of V2K and |
| | RNM as red wine to drink”. |
| 5. | Video of genitalia in syphilis patients |
| | with words or sentence :”Hey, this horrifying genitals |
| | want to fuck your operators using device of V2K and |
| 6. | Video of a small viper with words :” |
| | Hey, I want to stuff this horrifying small snake |
| | into your your operators' nose when you are using |
| | device of V2K and RNM “. |
| n . . . | And so on. |

All above seven examples can cause universal disgusting to every people, but can not insult anyone because the identification operating in private room.

FIG. 2A to FIG. 2F illustrate a first phase of flowchart.

Category b:

Category b is used for identifying a TP who is a victim of abuse technology of V2K and RNM.

The category b database contents which are specifically causing remote operator (ROD) using devices of V2K & RNM funny psychological response. E.g., LAUFHING, but they will not cause tested person's (TP's) funny psychological reaction, because the identification system administrator (ISA) lets tested person (TP) himself/herself choose some funny pictures or videos with words pointing directly at ROD, but do not pointing TP self.

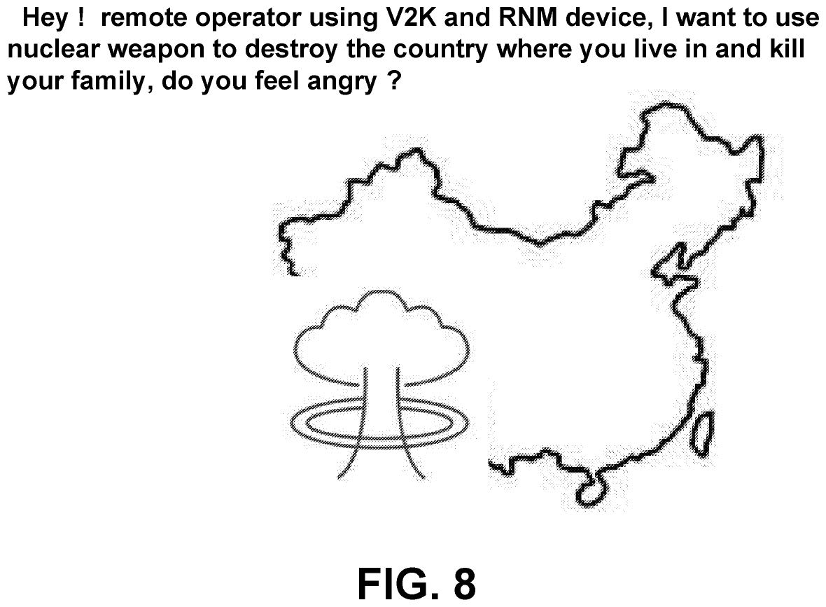

For example, A picture drawing two flies stopping on one's hair mating, in this picture has such words: “Hey, the flies couple are mating on your ROD's hair, do you feel funny? Please check FIG. 8.

After TP has chosen a kind of funny picture with teasing words to ROD from database b, he/she must answer the designed question which shows on a touch screen. Meanwhile, an advanced automatic guide system in PLC to show CORRECT or WRONG on every TP pressing button on the touch screen.

After category identification, an identification system administrator (ISA) connects PM to TP and then asks TP one question: ‘Did you answer these question on this touchscreen honestly?”, TP answer; “Yes, I did”. ISA checks PM to confirm TP's answer is true, then finish identification. In case of TP complete this phase is not honest, ISA had to ask TP to repeat this identification phase.

According automatic control theory, multiple TP's funny retinal image signal psychological attacking cycles will generate precise identification: the funny psychological response comes from ROD but does not belong to TP's.

Special PLC algorithm for identifying Funny reaction of V2K signal refer FIG. 15B.

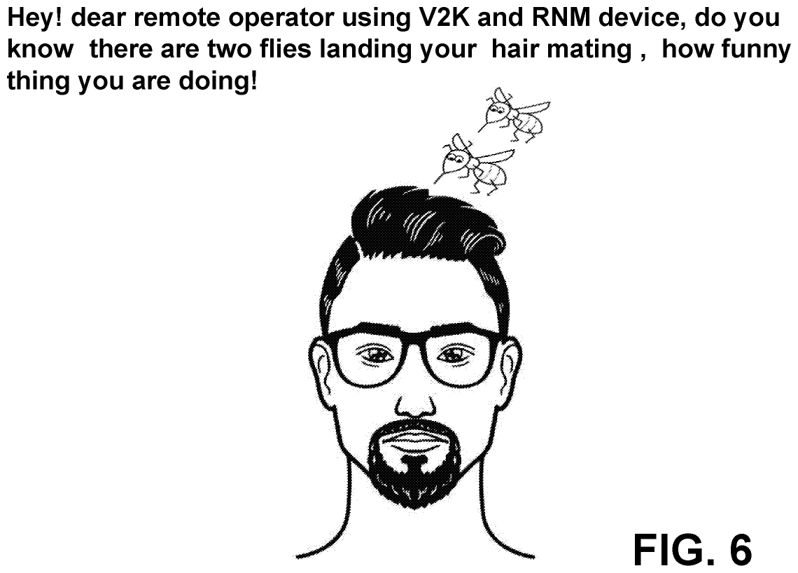

FIG. 2A to FIG. 2F illustrates a first phase of flowchart. FIG. 6 illustrates example of a picture with some words funny stories to tease ROD.

Funny Jokes Examples List

Category c:

| | |
|---|---|
| 1. | Two guys stole a calendar. They got six months each. |
| 2. | I make mistakes; I'll be the second to admit it. |
| 3. | MORNING TEA |
| | Question: What has T in the beginning, T in the middle, and T at the end? Answer: |
| | A teapot. |
| 4. | SICK SNOWMAN |
| | Question: What do snowmen do when they're not feeling well? Answer: They take |
| | a chill pill! |
| 5. | A PROPOSAL |
| | How did the telephone propose to its girlfriend? He gave her a ring. |
| 6. | ALL'S WE'LL THAT ENDS WE'LL |
| | We'll we'll we'll . . . if it isn't autocorrect. |
| n. | And so on . . . |

Category c is used for identifying ROD's political trend and religious belief.

The category c database contains all kinds of pictures or videos with some words attacking ROS's political trend and religious belief are specifically causing remote operator's (ROD's) using devices of V2K & RNM angry psychological response: angry curse and refutation, but they will not cause tested person's (TP's) angry psychological reaction, because the identification system administrator (ISA) lets tested person (TP) himself/herself choose some pictures or videos with some words only attacking ROD, that is these words only fiercely oppose ROD″s political trends or these words only seriously insult ROD's religious beliefs in this database intentionally, but these words does not attack TP. So these words only to cause ROD's angry psychological response, but does not cause the tested person (TP) himself/herself angry psychological reaction.

There is a more very clear example: we assume the TP is not a Communist Party member, but ROD is a Communist Party member who thinks that the Communist Party flag and Communist Party emblem are sacred and inviolable. Then ISA asks TP to choose a picture or a video to insult the Communist Party flag and Communist Party emblem with stinky stool to stain it or burn them, in this case, TP in the invention system will feel and sense ROD's angry via V2K technology on TP's auditory cortex, ISA is sure ROD is a Communist Party member, because people who are not Communist Party member feel nothing insulting the Communist Party flag and Communist Party emblem in such way, only a Communist Party member can feel angry, so there is not any psychologist can deny the accuracy of the invention of identification.

After TP has chosen a kind of picture with some words only attacking ROD from database c, he/she only need to watch these picture with words in a projecting system to launch a retinal image signals to attack ROD and then answer question in diamond frame in flowchart which shows on a touch screen,

Meanwhile, an advanced automatic guide system in PLC to show CORRECT or WRONG on every TP pressing button on the touch screen which guides TP to reach final identification fast.

After category identification, an identification system administrator (ISA) connects PM to TP and then asks TP one question: ‘Did you answer these question on this touchscreen honestly?”, TP answer; “Yes, I did”. ISA checks PM to confirm TP's answer is true, then finish identification. In case of TP complete this phase is not honest, ISA had to ask TP to repeat this identification phase.

According automatic control theory, multiple TP's political or religious watching pictures with words retinal image signals attack cycles will generate precise identification: the angry psychological response comes from RODs because of political or religious reasons, but does not belong to TP's, so ROD's political trend and religious trend are identified.

C. Special PLC algorithm for identifying ROD's political and religion belief refer FIG. 15C.

Political Ideologies Examples List

| | |
|---|---|
| 1. | absolutism. System where the rulers have unlimited control. |
| 2. | anarchism. Society without government, laws, police or other authority. System of |
| | self-control. |
| 3. | aristocracy. The privilege of social class whose members possess |
| | disproportionately large percentage of society's wealth, prestige and political |
| | influence. |
| 4. | autocracy. Supreme political power is in the hands of one person whose decision |
| | are unregulated.. |
| 5. | capitalism. Right-wing political system where the principle means of production |
| | and distribution are in private hands. |
| 6. | communism. Extreme left-wing ideology based on the revolutionary socialist |
| | teachings of Marx. Collective ownership and a planned economy. Each should |
| | work to their capability and receive according to their needs. |
| 7. | conservatism. Governmental system where the existing institution are maintained, |
| | emphasizing free-enterprise and minimal governmental intervention. |
| 8. | democracy. Government by the people usually through elected representatives. |
| 9. | dictatorship. Government by a single person with absolute control over the |
| | resources of the state. |
| 10. | egalitaranism. Belief where all citizens have equal rights and privileges. |
| 11. | fascism. Extreme right-wing ideology where the existing social order is protected |
| | by the forcible suppression of the working class. |
| 12. | imperialism. The extension of power and rule beyond established geographical |
| | boundaries. |
| 13. | liberalism. Representative government, free-speech, abolition of class privilege |
| | and state protection of the individual. |
| 14. | Marxism. Developed by Marx and Engles, it proposes that all is subject to change |
| | and resistance to change necessitates the overthrow of the system through class |
| | struggle. |
| 15. | Maoism. Interpretation of Marxist communism emphasizing the development of |
| | agriculture. |
| 16. | Monarchy. A form of rule in which the head of state is a King or Queen. |
| 17. | nationalism. The unification of the state and release from foreign rule. |
| 18. | oligarchy. A system of government in which virtually all power is held a small |
| | number of wealthy people who shape policy to benefit themselves. |
| 19. | populism. Collective noun for the ideologies which demand the redistribution of |
| | political power and economic leadership to the ‘common people’. |
| 20. | socialism. Left-wing political system where the principle means of production, |
| | distribution and exchange are in common ownership. |
| | theocracy. Rule by the church. |
| 21. | totalitarianism. Government control of all activities. |
| 22. | Trotskyism. Form of Marxism incorporating the concept of permanent revolution. |
| 23. | liberty. The freedom of speech and the right to dissent. |
| 24. | equality. A classless society with the redistribution of wealth through a welfare |
| | state. |
| 25. | fraternity. The communal brotherhood, working and living as one. |
| 26. | authority. The preservation of order through an evolved authority. |
| 27. | hierarchy. The continuation of the existing social order. |
| 28. | property. The right to private ownership |

Religious Belief List

| | | |
|---|---|---|
| 1. | Agnosticism | A form of belief, rather than a specific system. Belief that |
| | | God, if it exists, is by nature unknowable and will always be |
| | | unknowable |
| 2. | Amish | Peaceful Christian fundamentalist group famous for its |
| | | rejection of technology and strict adherence to OT and NT |
| | | laws |
| 3. | ancestor worship | A form of belief, rather than a specific system. Belief that |
| | | good relations need to be kept with tribal ancestor spirits. |
| | | Often a form of Shamanism |
| 4. | animism | A form of belief, rather than a specific system. The belief |
| | | that all objects contain spirits. More a traditional form of |
| | | belief than a “religion” in the Western sense |
| 5. | Brahma Kumari | This group are preparing to rule the world after a coming |
| | | apocalypse, and embrace many practices which are now |
| | | called New Age |
| 6. | Chinese Religion | A varied cultural religion practiced traditionally on a town- |
| | | by-town and region-by-region basis |
| 7. | Christadelphians | Bible-based Christianity |
| 7. | Confucianism | A world religion 10, 11. A collection of ethical and moral |
| | | teachings |
| 8. | Divine Lightmission | A self-help religious group involving meditation, with few |
| | | fixed beliefs |
| 9. | Druze | A semi-secretive esoteric religion with features of a Mystery |
| | | Religion |
| 10. | dualism | A form of belief, rather than a specific system. Belief that |
| | | either (1) There is a good and evil god of equal, or almost- |
| | | equal power, or (2) there are two gods, such as a male and |
| | | female one |
| 11. | Hare Krishna | The embracing of Krishna through correct living, honesty, |
| | | spiritual life and austerity |
| 12. | Jehovah's | A 140-year-old Christian fundamentalist/literalist |
| | Witnesses | organisation famous for preaching that the world is about to |
| | | end |
| 13. | Mennonite | A Protestant Christian denomination |
| 14. | Native American | Beliefs vary from tribe to tribe and are sometimes noticeably |
| | Church | Christian |
| 15. | occultism | A form of belief, rather than a specific system. Normally |
| | | existing within other belief systems, occult systems |
| | | concentrate on esoteric meanings in texts, often with magical |
| | | undertones |
| 16. | polytheism | A form of belief, rather than a specific system. Belief in |
| | | multiple gods, often in some form of hierarchy |
| 17. | Rastafarian | God (called Jah) fathered a black Jesus; marijuana use in |
| | | rituals |
| 18. | Salvation Army | Christian organisation of evangelists organised along |
| | | military lines, famous for charity work |
| 19. | Santería | A combination of West African, Caribbean beliefs with |
| | | some elements of Roman Catholicism |
| 20. | Theosophy | Mired in fraud arrests and exposés, the Spiritualism scene |
| | | had soured, so Madame Blavatsky reinvented her routine as |
| | | a new religion, using an Indian theme |
| 21. | Unification Church | Sun Myung Moon embodied the Second Coming of Christ, |
| | | and his commercialist church runs a media empire |
| 22. | Unitas Fratrum | A Protestant Christian denomination |
| 23. | Witchcraft | A description of various cultural practices, which are often |
| | | part of a parent belief system |

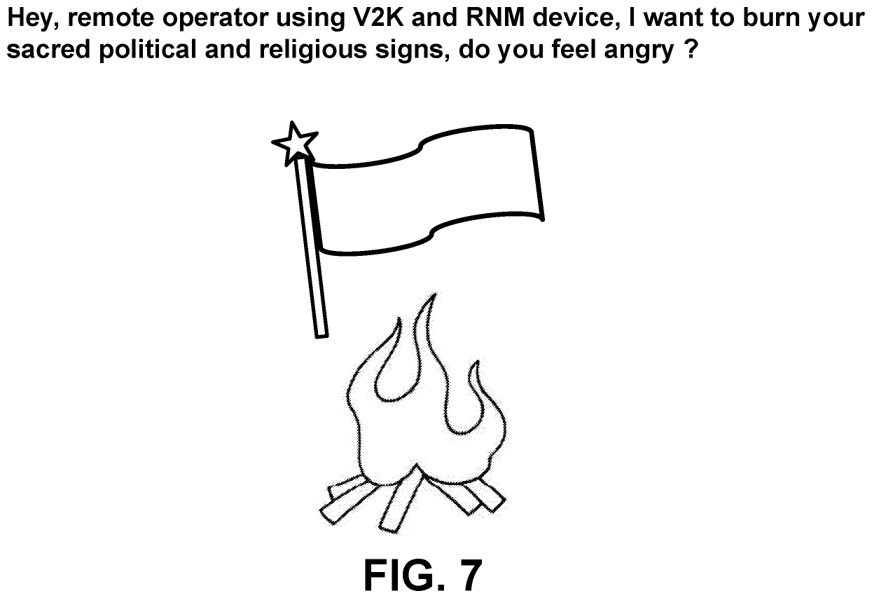

FIG. 3A to FIG. 3E illustrates a second phase of flowchart. FIG. 7 illustrates illustrates an examples of picture with some words to burn ROD's political and religious sign.

Category d:

Category d is used for identifying ROD's nationality and race. picture with some words to burn ROD's political and religious sign.

The category d database contents are different countries pictures and different races pictures or videos with some words only used attacking ROD's country and ROD's race which specifically cause remote operator's (ROD's) using devices of V2K and RNM angry psychological response: angry curse, because the identification system administrator (ISA) lets tested person (TP) himself/herself choose some pictures or videos with some words which destroy ROD's country and ROD's race in this database intentionally only to cause ROD angry psychological response, but does not cause the tested person (TP) himself/herself angry psychological reaction.

For example, TP choose some nuclear explosion videos with words in database d, these words like such: “Hey, remote operator, I hope to use the nuclear weapon to destroy your country and kill all your family members, let your ROD's body and corpse became vapor or steam in high temperature, kill all your race from the earth”, such TP's retinal image signals psychological attack is sure to cause ROD's angry response which TP can feel and sense via V2K technology on TP's auditory cortex. Once ROD's angry is identified, ROD's nationality and race have been identified.

For example: TP can choose a video in which a huge earthquake destroying a country with some words like “Hey, dear remote operator, the huge earthquake will destroy your country and your family” from computer database, a country where RODs live to cause ROD's antipathy and angry which TP can feel clearly via V2K technology on TP's auditory cortex.

For example: TP can choose a huge tsunami drowning a country video with some words like “Hey, remote operator using V2K technology, the huge tsunami will destroy your ROD's country and drown your ROD's family” from computer database d to launch a retinal image signal psychological attack a country where RODs live to cause ROD's antipathy and angry which TP can feel clearly via V2K technology on TP's auditory cortex.

After TP has chosen a kind of content from database d, he/she only need to watch the video or picture with some words only attacking ROD in a computer to launch a retinal image signal psychological attack ROD and then answer question in diamond frame in flowchart which shows on touch screen.

Meanwhile, an advanced automatic guide system in PLC to show CORRECT or WRONG on every TP pressing button on the touch screen which guides TP to reach final identification fast.

After category identification, an identification system administrator (ISA) connects PM to TP and then asks TP one question: ‘Did you answer these question on this touchscreen honestly?”, TP answer; “Yes, I did”. ISA checks PM to confirm TP's answer is true, then finish identification. In case of TP complete this phase is not honest, ISA had to ask TP to repeat this identification phase.

According automatic control theory, multiple TP's retinal image signal psychological attack cycles will generate precise identification: the angry psychological response comes from ROD, because ROD's nationality or race are attacked and ROD are infuriated, but these does not belong to TP's.

Special PLC algorithm for identifying ROD's nationality and race refer FIG. 15D.

Because there are too much countries and races, so the list of countries and races omit here, when the machine puts into running, puts counties and races in database.

FIG. 3A to FIG. 3 E illustrates a second phase of flowchart. FIG. 8 illustrates an examples of a picture with some words a mushroom cloud of nuclear explosion rises on the land of a particular country.

Category e: Category e is used for identifying a TP is a victim of abuse technology of RNM & V2K.

The category e database contents with all kinds of foreign terrorist organizations are specifically causing remote operator's using devices of V2K & RNM ROD's angry psychological response: strongly deny, because the identification system administrator (ISA) lets tested person (TP) himself/herself choose pictures with some words which accuse RODs committed a terrorist crime, such as terrorist attack on United States and RODs belong to some terrorist organization to cause ROD's strongly deny which TP can feel and sense via V2K technology on TP's auditory cortex. Because everyone knows any terrorist who attacked USA, US government must eliminate the terrorist in anywhere in the world.

On the other hand, TP is not a terrorist and never commit felony, so TP does not need to deny or admit. So in this step, TP answer will choose NotYES&NotNO button, but TP is not willing top press YES button on the touchscreen more than ten times deliberately, but sure ROD will deny it firmly.

So once RODs make a strongly deny, it is sure the response from RODs and it is sure the response is not auditory hallucination belong the victim (TP). This is an important step to identify a victim of abusing technology of V2K & RNM.

After TP has chosen a kind of picture with such words from database e, he/she only need to watch the picture or video with such words to launch a retinal image signal psychological attack to ROD and then answer question in diamond frame in flowchart

Meanwhile, an advanced automatic guide system in PLC to show CORRECT or WRONG on every TP pressing button on the touch screen which guides TP to reach final identification fast.

After category identification, an identification system administrator (ISA) connects PM to TP and then asks TP one question: ‘Did you answer these question on this touchscreen honestly?”, TP answer; “Yes, I did”. ISA checks PM to confirm TP's answer is true, then finish this identification. In case of TP complete this phase is not honest, ISA had to ask TP to repeat this identification phase.

According automatic control theory, multiple TP's accusing ROD's belong terrorist organizations or convicted terrorist crime psychological attacking cycles will generate precise identification: the strongly denying psychological response comes from ROD, but these does not belong to TP's.

Special PLC algorithm for identifying a TP is a victim of abuse technology of V2K and RNM refer FIG. 15D.

FIG. 2A to FIG. 2F illustrates a first phase of flowchart.

Foreign organizations will be searched from time to time and added into the database e by ISA when in need.

Part Examples of Foreign Terrorist Organizations List with Date Designed

| | |
|---|---|
| 1. | Oct. 8, 1997 Abu Sayyaf Group (ASG) |
| 2. | Oct. 8, 1997 Aum Shinrikyo (AUM) |
| 3. | Oct. 8, 1997 Basque Fatherland and Liberty (ETA) |
| 4. | Oct. 8, 1997 Gama' a al-Islamiyya (Islamic Group - IG) |
| 5. | Oct. 8, 1997 HAMAS |
| 6. | Oct. 8, 1997 Harakat ul-Mujahidin (HUM) |
| 8. | Oct. 8, 1997 Hizballah |
| 8. | Oct. 8, 1997 Liberation Tigers of Tamil Eelam (LTTE) |
| 9. | Oct. 8, 1997 National Liberation Army (ELN) |
| 10. | Oct. 8, 1999 al-Qa' ida (AQ) |
| 11. | Mar. 27, 2002 Al-Aqsa Martyrs Brigade (AAMB) |
| 12. | Mar. 27, 2002 al-Qaida in the Islamic Maghreb (AQIM) |
| 13. | Feb. 17, 2004 Islamic State of Iraq and the Levant (formerly al-Qa' ida in Iraq) |
| 14. | Jan. 19, 2010 al-Qa' ida in the Arabian Peninsula (AQAP) |
| 15. | May 23, 2011 Army of Islam (AOI) |
| 16. | Nov. 14, 2013 Boko Haram |
| 17. | May 15, 2014 al-Nusrah Front |
| N. | And so on . . . |

Category f: Category f is used for identifying ROD's scope of language and dialect.

The category f database contains pictures and videos with different languages words and different dialects words to send retinal image signals to remote operator (ROD) for specifically causing remote operator's (ROD's) using devices of V2K and RNM psychological response: there are two cases. If the ROD knows the words language in the same drawing, ROD will make psychological response, so this can identify ROD know this language. On the other hand, if ROD do not know the words language, ROD will lighten harassment or stop harassment when they meet some foreign language or dialect which they do not know, because these ROD need time to understand and consider how response on their device of V2K & RNM on ROD using device.

For example, there are five pictures have same drawing with different languages; The drawing describes remote operator pissing pans and the urine is wet a lot. If a ROD meets one of the following five kinds of words language which he/she knows, The ROD will make a psychological response. If a ROD meets one of the following five kinds of words language he/she does not know, the ROD will not make a psychological response and lighten or stop his/her harassment. So ISA ask a TP to watch the five same drawing pictures with different words language in turn, the TP is waiting for which picture with obviously psychological response from a ROD with V2K feeling in TP's auditory cortex.

FIG. 10 Five examples of identifying ROD″s language scope list.

After TP has chosen a kind of picture with some words from database f, he/she has sent a retina image signal to ROD and made a psychological attack to ROD and then TP answer question in diamond frame in flowchart which shows on touch screen.

Meanwhile, an advanced automatic guide system in PLC to show CORRECT or WRONG on every TP pressing button on the touch screen which guides TP to reach final identification fast.

After category identification, an identification system administrator (ISA) connects PM to TP and then asks TP one question: ‘Did you answer these question on this touchscreen honestly?”, TP answer; “Yes, I did”. ISA checks PM to confirm TP's answer is true, then finish identification. In case of TP complete this phase is not honest, ISA had to ask TP to repeat this identification phase.

According automatic control theory, multiple TP's retina image signal psychological attacking cycles with words languages or words dialect will generate precise identification: ROD know what language and does not know what language.

Special PLC algorithm for identifying ROD's scale of language and dialect is similar to parts a,b,c, d,e, so here omit.

FIG. 3A to FIG. 3E illustrate a second phase of flowchart.

Table 1 shows examples of languages spoken in different countries of the world.

Languages Spoken in Different Countries of the World

| | Languages spoken (the percent of the population that speaks a |
|---|---|
| Country | particular language is also given, if available) |
| Afghanistan | Dari Persian, Pashtu (both official), other Turkic and minor |
| | languages |
| Albania | Albanian (Tosk is the official dialect), Greek |
| Algeria | Arabic (official), French, Berber dialects |
| Andorra | Catalán (official), French, Castilian, Portuguese |
| Angola | Portuguese (official), Bantu and other African languages |
| Antigua and | English (official), local dialects |
| Barbuda | |
| Argentina | Spanish (official), English, Italian, German, French |
| Armenia | Armenian 98%, Yezidi, Russian |
| Australia | English 79%, native and other languages |
| Austria | German (official nationwide); Slovene, Croatian, Hungarian |
| | (each official in one region) |
| Azerbaijan | Azerbaijani Turkic 89%, Russian 3%, Armenian 2%, other 6% |
| | (1995 est.) |
| Bahamas | English (official), Creole (among Haitian immigrants) |
| Bahrain | Arabic, English, Farsi, Urdu |
| Bangladesh | Bangla (official), English |
| Barbados | English |
| Belarus | Belorussian (White Russian), Russian, other |
| Belgium | Dutch (Flemish) 60%, French 40%, German less than 1% (all |
| | official) |
| Belize | English (official), Spanish, Mayan, Garifuna (Carib), Creole |
| Benin | French (official), Fon, Yoruba, tribal languages |
| Bhutan | Dzongkha (official), Tibetan dialects (among Bhotes), |
| | Nepalese dialects (among Nepalese) |
| Bolivia | Spanish, Quechua, Aymara (all official) |
| Bosnia and | Bosnian, Croatian, Serbian |
| Herzegovina | |
| Botswana | English 2% (official), Setswana 78%, Kalanga 8%, |
| | Sekgalagadi 3%, other (2001) |
| Brazil | Portuguese (official), Spanish, English, French |
| Brunei | Malay (official), English, Chinese |
| Bulgaria | Bulgarian 85%, Turkish 10%, Roma 4% |
| Burkina Faso | French (official); native African (Sudanic) languages 90% |
| Burundi | Kirundi and French (official), Swahili |
| Cambodia | Khmer 95% (official), French, English |
| Cameroon | French, English (both official); 24 major African language |
| | groups |
| Canada | English 59.3%, French 23.2% (both official); other 17.5% |
| Cape Verde | Portuguese, Criuolo |
| Central African | French (official), Sangho (lingua franca, national), tribal |
| Republic | languages |
| Chad | French, Arabic (both official); Sara; more than 120 languages |
| | and dialects |
| Chile | Spanish |
| China | Standard Chinese (Mandarin/Putonghua), Yue (Cantonese), Wu |
| | (Shanghaiese), Minbei (Fuzhou), Minnan (Hokkien- |
| | Taiwanese), Xiang, Gan, Hakka dialects, minority languages |
| Colombia | Spanish |
| Comoros | Arabic and French (both official), Shikomoro (Swahili/Arabic |
| | blend) |
| Congo, Democratic | French (official), Lingala, Kingwana, Kikongo, Tshiluba |
| Republic of the | |
| Congo, Republic of | French (official), Lingala, Monokutuba, Kikongo, many local |
| | languages and dialects |
| Costa Rica | Spanish (official), English |
| Côte d'Ivoire | French (official) and African languages (Dioula esp.) |
| Croatia | Croatian 96% (official), other 4% (including Italian, |
| | Hungarian, Czech, Slovak, German) |
| Cuba | Spanish |
| Cyprus | Greek, Turkish (both official); English |
| Czech Republic | Czech |
| Denmark | Danish, Faroese, Greenlandic (Inuit dialect), German; English |
| | is the predominant second language |
| Djibouti | French and Arabic (both official), Somali, Afar |
| Dominica | English (official) and French patois |
| Dominican Republic | Spanish |
| East Timor | Tetum, Portuguese (official); Bahasa Indonesia, English; other |
| | indigenous languages, including Tetum, Galole, Mambae, and |
| | Kemak |
| Ecuador | Spanish (official), Quechua, other Amerindian languages |
| Egypt | Arabic (official), English and French widely understood by |
| | educated classes |
| El Salvador | Spanish, Nahua (among some Amerindians) |
| Equatorial Guinea | Spanish, French (both official); pidgin English, Fang, Bubi, Ibo |
| Eritrea | Afar, Arabic, Tigre and Kunama, Tigrinya, other Cushitic |
| | languages |
| Estonia | Estonian 67% (official), Russian 30%, other (2000) |
| Ethiopia | Amharic, Tigrigna, Orominga, Guaragigna, Somali, Arabic, |
| | English, over 70 others |
| Fiji | English (official), Fijian, Hindustani |
| Finland | Finnish 92%, Swedish 6% (both official); small Sami- (Lapp) |
| | and Russian-speaking minorities |
| France | French 100%, rapidly declining regional dialects (Provençal, |
| | Breton, Alsatian, Corsican, Catalan, Basque, Flemish) |
| Gabon | French (official), Fang, Myene, Nzebi, Bapounou/Eschira, |
| | Bandjabi |
| Gambia | English (official), Mandinka, Wolof, Fula, other indigenous |
| Georgia | Georgian 71% (official), Russian 9%, Armenian 7%, |
| | Azerbaijani 6%, other 7% (Abkhaz is the official language in |
| | Abkhazia) |
| Germany | German |
| Ghana | English (official), African languages (including Akan, Moshi- |
| | Dagomba, Ewe, and Ga) |
| Greece | Greek 99% (official), English, French |
| Grenada | English (official), French patois |
| Guatemala | Spanish 60%, Amerindian languages 40% (23 officially |
| | recognized Amerindian languages, including Quiche, |
| | Cakchiquel, Kekchi, Mam, Garifuna, and Xinca) |
| Guinea | French (official), native tongues (Malinké, Susu, Fulani) |
| Guinea-Bissau | Portuguese (official), Criolo, African languages |
| Guyana | English (official), Amerindian dialects, Creole, Hindi, Urdu |
| Haiti | Creole and French (both official) |
| Honduras | Spanish (official), Amerindian dialects; English widely spoken |
| | in business |
| Hungary | Magyar (Hungarian) 94%, other 6% |
| Iceland | Icelandic, English, Nordic languages, German widely spoken |
| India | Hindi 30%, English, Bengali, Gujarati, Kashmiri, Malayalam, |
| | Marathi, Oriya, Punjabi, Tamil, Telugu, Urdu, Kannada, |
| | Assamese, Sanskrit, Sindhi (all official); Hindi/Urdu; 1,600+ |
| | dialects |
| Indonesia | Bahasa Indonesia (official), English, Dutch, Javanese, and |
| | more than 580 other languages and dialects |
| Iran | Persian and Persian dialects 58%, Turkic and Turkic dialects |
| | 26%, Kurdish 9%, Luri 2%, Balochi 1%, Arabic 1%, Turkish |
| | 1%, other 2% |
| Iraq | Arabic (official), Kurdish (official in Kurdish regions), |
| | Assyrian, Armenian |
| Ireland | English, Irish (Gaelic) (both official) |
| Israel | Hebrew (official), Arabic, English |
| Italy | Italian (official); German-, French-, and Slovene-speaking |
| | minorities |
| Jamaica | English, Jamaican Creole |
| Japan | Japanese |
| Jordan | Arabic (official), English |
| Kazakhstan | Kazak (Qazaq, state language) 64%; Russian (official, used in |
| | everyday business) 95% (2001 est.) |
| Kenya | English (official), Swahili (national), and numerous indigenous |
| | languages |
| Kiribati | English (official), I-Kiribati (Gilbertese) |
| Korea, North | Korean |
| Korea, South | Korean, English widely taught |
| Kosovo | Albanian (official), Serbian (official), Bosnian, Turkish, Roma |
| Kuwait | Arabic (official), English |
| Kyrgyzstan | Kyrgyz, Russian (both official) |
| Laos | Lao (official), French, English, various ethnic languages |
| Latvia | Latvian 58% (official), Russian 38%, Lithuanian, other (2000) |
| Lebanon | Arabic (official), French, English, Armenian |
| Lesotho | English, Sesotho (both official); Zulu, Xhosa |
| Liberia | English 20% (official), some 20 ethnic-group languages |
| Libya | Arabic, Italian, and English widely understood in major cities |
| Liechtenstein | German (official), Alemannic dialect |
| Lithuania | Lithuanian 82% (official), Russian 8%, Polish 6% (2001) |
| Luxembourg | Luxermbourgish (national) French, German (both |
| | administrative) |
| Macedonia | Macedonian 67%, Albanian 25% (both official); Turkish 4%, |
| | Roma 2%, Serbian 1% (2002) |
| Madagascar | Malagasy and French (both official) |
| Malawi | Chichewa 57.2% (official), Chinyanja 12.8%, Chiyao 10.1%, |
| | Chitumbuka 9.5%, Chisena 2.7%, Chilomwe 2.4%, Chitonga |
| | 1.7%, other 3.6% (1998) |
| Malaysia | Bahasa Melayu (Malay, official), English, Chinese dialects |
| | (Cantonese, Mandarin, Hokkien, Hakka, Hainan, Foochow), |
| | Tamil, Telugu, Malayalam, Panjabi, Thai; several indigenous |
| | languages (including Iban, Kadazan) in East Malaysia |
| Maldives | Maldivian Dhivehi (official); English spoken by most |
| | government officials |
| Mali | French (official), Bambara 80%, numerous African languages |
| Malta | Maltese and English (both official) |
| Marshall Islands | Marshallese 98% (two major dialects from the Malayo- |
| | Polynesian family), English widely spoken as a second |
| | language (both official); Japanese |
| Mauritania | Hassaniya Arabic (official), Pulaar, Soninke, French, Wolof |
| Mauritius | English less than 1% (official), Creole 81%, Bojpoori 12%, |
| | French 3% (2000) |
| Mexico | Spanish, various Mayan, Nahuatl, and other regional |
| | indigenous languages |
| Micronesia | English (official, common), Chukese, Pohnpeian, Yapase, |
| | Kosrean, Ulithian, Woleaian, Nukuoro, Kapingamarangi |
| Moldova | Moldovan (official; virtually the same as Romanian), Russian, |
| | Gagauz (a Turkish dialect) |
| Monaco | French (official), English, Italian, Monégasque |
| Mongolia | Mongolian, 90%; also Turkic and Russian (1999) |
| Montenegro | Serbian/Montenegrin (Ijekavian dialect-official) |
| Morocco | Arabic (official), Berber dialects, French often used for |
| | business, government, and diplomacy |
| Mozambique | Portuguese 9% (official; second language of 27%), Emakhuwa |
| | 26%, Xichangana 11%, Elomwe 8%, Cisena 7%, Echuwabo |
| | 6%, other Mozambican languages 32% (1997) |
| Myanmar | Burmese, minority languages |
| Namibia | English 7% (official), Afrikaans is common language of most |
| | of the population and of about 60% of the white population, |
| | German 32%; indigenous languages: Oshivambo, Herero, |
| | Nama |
| Nauru | Nauruan (official), English |
| Nepal | Nepali 48% (official), Maithali 12%, Bhojpuri 7%, Tharu 6%, |
| | Tamang 5%, others. English spoken by many in government |
| | and business (2001) |
| Netherlands | Dutch, Frisian (both official) |
| New Zealand | English, Maori (both official) |
| Nicaragua | Spanish 98% (official); English and indigenous languages on |
| | Atlantic coast (1995) |
| Niger | French (official), Hausa, Djerma |
| Nigeria | English (official), Hausa, Yoruba, Ibo, Fulani, and more than |
| | 200 others |
| Norway | Bokmål Norwegian, Nynorsk Norwegian (both official); small |
| | Sami- and Finnish-speaking minorities (Sami is official in six |
| | municipalities) |
| Oman | Arabic (official), English, Baluchi, Urdu, Indian dialects |
| Pakistan | Urdu 8%, English (both official); Punjabi 48%, Sindhi 12%, |
| | Siraiki (a Punjabi variant) 10%, Pashtu 8%, Balochi 3%, |
| | Hindko 2%, Brahui 1%, Burushaski, and others 8% |
| Palau | Palauan 64.7%, English 9.4%, Sonsoralese, Tobi, Angaur (each |
| | official on some islands), Filipino 13.5%, Chinese 5.7%, |
| | Carolinian 1.5%, Japanese 1.5%, other Asian 2.3%, other |
| | languages 1.5% (2000) |
| Palestinian State | Arabic, Hebrew, English |
| (proposed) | |
| Panama | Spanish (official), English 14%, many bilingual |
| Papua New Guinea | Tok Pisin (Melanesian Pidgin, the lingua franca), Hiri Motu (in |
| | Papua region), English 1%-2%; 715 indigenous languages |
| Paraguay | Spanish, Guaraní (both official) |
| Peru | Spanish, Quéchua (both official); Aymara; many minor |
| | Amazonian languages |
| Philippines | Filipino (based on Tagalog), English (both official); eight |
| | major dialects: Tagalog, Cebuano, Ilocano, Hiligaynon or |
| | Ilonggo, Bicol, Waray, Pampango, and Pangasinense |
| Poland | Polish 98% (2002) |
| Portugal | Portuguese (official), Mirandese (official, but locally used) |
| Qatar | Arabic (official); English a common second language |
| Romania | Romanian (official), Hungarian, German |
| Russia | Russian, others |
| Rwanda | Kinyarwanda, French, and English (all official); Kiswahili in |
| | commercial centers |
| St. Kitts and Nevis | English |
| St. Lucia | English (official), French patois |
| St. Vincent and the | English, French patois |
| Grenadines | |
| Samoa | Samoan, English |
| San Marino | Italian |
| São Tomé | Portuguese (official) |
| and Príncipe | |
| Saudi Arabia | Arabic |
| Senegal | French (official); Wolof, Pulaar, Jola, Mandinka |
| Serbia | Serbian (official); Romanian, Hungarian, Slovak, and Croatian |
| | (all official in Vojvodina); Albanian (official in Kosovo) |
| Seychelles | Seselwa Creole 92%, English 5%, French (all official) (2002) |
| Sierra Leone | English (official), Mende (southern vernacular), Temne |
| | (northern vernacular), Krio (lingua franca) |
| Singapore | Mandarin 35%, English 23%, Malay 14.1%, Hokkien 11.4%, |
| | Cantonese 5.7%, Teochew 4.9%, Tamil 3.2%, other Chinese |
| | dialects 1.8%, other 0.9% (2000) |
| Slovakia | Slovak 84% (official), Hungarian 11%, Roma 2%, Ukrainian |
| | 1% (2001) |
| Slovenia | Slovenian 91%, Serbo-Croatian 5% (2002) |
| Solomon Islands | English 1%-2% (official), Melanesian pidgin (lingua franca), |
| | 120 indigenous languages |
| Somalia | Somali (official), Arabic, English, Italian |
| South Africa | IsiZulu 23.8%, IsiXhosa 17.6%, Afrikaans 13.3%, Sepedi |
| | 9.4%, English 8.2%, Setswana 8.2%, Sesotho 7.9%, Xitsonga |
| | 4.4%, other 7.2% |
| South Sudan | English (official), Arabic (includes Juba and Sudanese variants) |
| | (official), regional languages include Dinka, Nuer, Bari, Zande, |
| | Shilluk |
| Spain | Castilian Spanish 74% (official nationwide); Catalan 17%, |
| | Galician 7%, Basque 2% (each official regionally) |
| Sri Lanka | Sinhala 74% (official and national), Tamil 18% (national), |
| | other 8%; English is commonly used in government and spoken |
| | competently by about 10% |
| Sudan | Arabic (official), Nubian, Ta Bedawie, diverse dialects of |
| | Nilotic, Nilo-Hamitic, Sudanic languages, English |
| Suriname | Dutch (official), Surinamese (lingua franca), English widely |
| | spoken, Hindustani, Javanese |
| Swaziland | English, siSwati (both official) |
| Sweden | Swedish, small Sami- and Finnish-speaking minorities |
| Switzerland | German 64%, French 20%, Italian 7% (all official); Romansch |
| | 0.5% (national) |
| Syria | Arabic (official); Kurdish, Armenian, Aramaic, Circassian |
| | widely understood; French, English somewhat understood |
| Taiwan | Chinese (Mandarin, official), Taiwanese (Min), Hakka dialects |
| Tajikistan | Tajik (official), Russian widely used in government and |
| | business |
| Tanzania | Swahili, English (both official); Arabic; many local languages |
| Thailand | Thai (Siamese), English (secondary language of the elite), |
| | ethnic and regional dialects |
| Togo | French (official, commerce); Ewé, Mina (south); Kabyé, |
| | Dagomba (north); and many dialects |
| Tonga | Tongan (an Austronesian language), English |
| Trinidad and | English (official), Hindi, French, Spanish, Chinese |
| Tobago | |
| Tunisia | Arabic (official, commerce), French (commerce) |
| Turkey | Turkish (official), Kurdish, Dimli, Azeri, Kabardian |
| Turkmenistan | Turkmen 72%; Russian 12%; Uzbek 9%, other 7% |
| Tuvalu | Tuvaluan, English, Samoan, Kiribati (on the island of Nui) |
| Uganda | English (official), Ganda or Luganda, other Niger-Congo |
| | languages, Nilo-Saharan languages, Swahili, Arabic |
| Ukraine | Ukrainian 67%, Russian 24%, Romanian, Polish, Hungarian |
| United Arab | Arabic (official), Persian, English, Hindi, Urdu |
| Emirates | |
| United | English, Welsh, Scots Gaelic |
| Kingdom | |
| United States | English 82%, Spanish 11% (2000) |
| Uruguay | Spanish, Portunol, or Brazilero |
| Uzbekistan | Uzbek 74.3%, Russian 14.2%, Tajik 4.4%, other 7.1% |
| Vanuatu | Bislama 23% (a Melanesian pidgin English), English 2%, |
| | French 1% (all 3 official); more than 100 local languages 73% |
| Vatican City (Holy | Italian, Latin, French, various other languages |
| See) | |
| Venezuela | Spanish (official), numerous indigenous dialects |
| Vietnam | Vietnamese (official); English (increasingly favored as a |
| | second language); some French, Chinese, Khmer; mountain |
| | area languages (Mon-Khmer and Malayo-Polynesian) |
| Western Sahara | Hassaniya Arabic, Moroccan Arabic |
| (proposed state) | |
| Yemen | Arabic |
| Zambia | English (official); major vernaculars: Bemba, Kaonda, Lozi, |
| | Lunda, Luvale, Nyanja, Tonga; about 70 other indigenous |
| | languages |
| Zimbabwe | English (official), Shona, Ndebele (Sindebele), numerous |
| | minor tribal dialects |

Category g: Category g is used for identifying ROD's scope of scientific knowledge.

The category g database has different pictures or videos of scientific knowledge with some words specifically test ROD if the ROD know or understand the scientific knowledge? If the ROD know or understand scientific knowledge, he/she will make some psychological response which a TP can feel or sense via V2K technology on TP's auditory cortex; On the other hand, if the ROD does not know or understand the scientific knowledge, the ROD will slow down psychological response: lighten harassment or stop harassment, because the ROD need time to consider how to response.

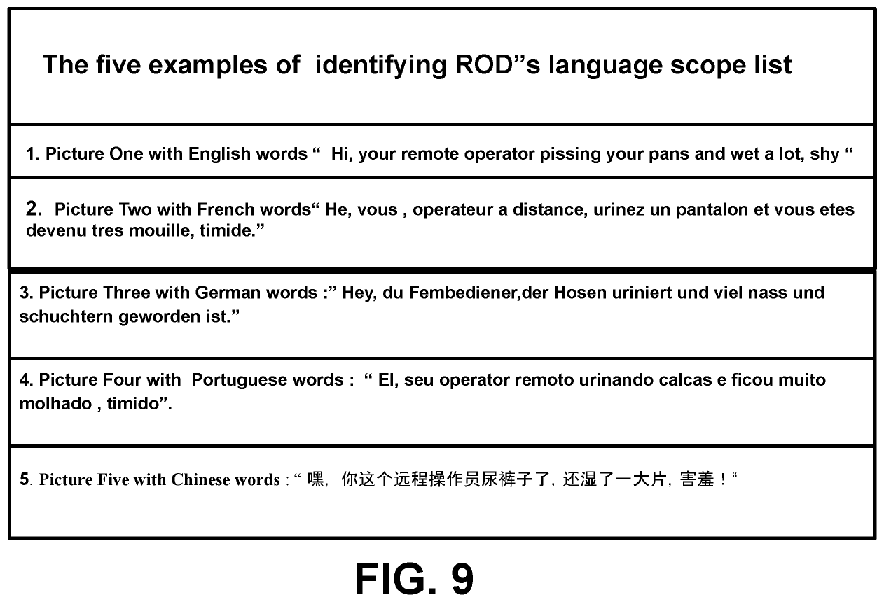

For examples, we assume a TP has computer technology knowledge and know how change theses data among hexadecimal, binary and decimal. At this point, TP can choose a picture like following: Please check FIG. 9.

In this case, if the ROD know the question, the ROD will response. On the other hand, if the ROD does not know the question, the ROD will slow down the harassment, even stop harassment to TP, which TP can feel or sense via V2K technology effecting on TP's auditory cortex.

When these questions are processing, we can find an obvious phenomenon: TP feel or sense the remote harassment get weaken or even disappeared temporarily, the reason is that those ROD do not understand how change data among hexadecimal, binary and decimal, so those remote operators (ROD) have no way to response on the device of V2K & RNM as soon as possible.

After TP has chosen a kind of picture with such words from database g, he/she only need to watch the picture with such words to launch a retinal image signal psychological attack to ROD and then answer question in diamond frame in flowchart which shows on touch screen.

Meanwhile, an advanced automatic guide system in PLC to show CORRECT or WRONG on every TP pressing button on the touch screen which guides TP to reach final identification fast.

After category identification, an identification system administrator (ISA) connects PM to TP and then asks TP one question: ‘Did you answer these question on this touchscreen honestly?”, TP answer; “Yes, I did”. ISA checks PM to confirm TP's answer is true, then finish identification. In case of TP complete this phase is not honest, ISA had to ask TP to repeat this identification phase.

According automatic control theory, multiple TP's such retinal image signal psychological attacking cycles with scientific knowledge will generate precise identification: ROD's scientific knowledge scale. FIG. 9 illustrates an examples of a picture with some words to test ROD know what scientific and technical knowledge.

Special PLC algorithm for identifying ROD's scale of scientific knowledge is similar to parts a,b,c,d,e, so here omit.

FIG. 3A to FIG. 3E illustrate a second phase of flowchart.

Table 2 shows examples of different kinds of sciences.

List of Different Kinds of Sciences

| Name of Sciences | Description |
|---|---|
| Acoustics | The study of sound (or the science of sound) |
| Aerodynamics | The study of the motion and control of solid bodies like aircraft, |
| | missiles, etc., in air |
| Aeronautics | The science or art of flight |
| Aeronomy | The study of the earth's upper atmosphere, including its |
| | composition, density, temperature and chemical reactions, as |
| | recorded by sounding rockets and earth satellites |
| Aerostatics | The branch of statics that deals with gases in equilibrium and with |
| | gases and bodies in them |
| Aetiology | The science of causation |
| Agrobiology | The science of plant life and plant nutrition |
| Agronomy | The science of soil management and the production of field crops |
| Agrostology | The study of grasses |
| Alchemy | Chemistry in ancient times |
| Anatomy | The science dealing with the structure of animals, plants or human |
| | body |
| Anthropology | The science that deals with the origins, physical and cultural |
| | development of mankind |
| Arboriculture | Cultivation of trees and vegetables |
| Archaeology | The study of antiquities |
| Astrochemistry | The study of interstellar matter with a view to knowing the origin |
| | of universe |
| Astrology | The ancient art of predicting the course of human destinies with |
| | the help of indications deduced from the position and movement |
| | of the heavenly bodies |
| Astronautics | The science of space travel |
| Astronomy | The study of the heavenly bodies |
| Astrophysics | The branch of astronomy concerned with the physical nature of |
| | heavenly bodies |
| Autoecology | The study deals with the ecology of species |
| Bacteriology | The study of bacteria |
| Biochemistry | The study of chemical processes of living things |
| Bioclimatology | Studies the effects of climate upon living organisms |
| Biology | The study of living things |
| Biometry | The application of mathematics to the study of living things |
| Biomechanics | The study of the mechanical laws relating to the movement or |
| | structure of living organisms |
| Biometeorology | Studies the effects of atmospheric conditions on living organisms |
| Bionics | The study of functions, characteristics and phenomena observed in |
| | the living world and the application of this knowledge to the world |
| | of machines |
| Bionomics | The study of the relation of an organism to its environments |
| Bionomy | The science of the laws of life |
| Biophysics | The physics of vital processes (living things) |
| Botany | The study of plants |
| Ceramics | The art and technology of making objects from clay, etc. (pottery) |
| Chemistry | The study of elements and their laws of combination and |
| | behaviour |
| Chemotherpy | The treatment of disease by using chemical substances |
| Chronobiology | The study of the duration of life |
| Chronology | The science of arranging time in periods and ascertaining the dates |
| | and historical order of past events |
| Climatotherapy | The treatment of disease through suitable climatic environment, |
| | often, but not always, found in recognised health resorts. As |
| | climate is subject to seasonal variations, the required environment |
| | may have to be sought in different localities at different periods of |
| | the year |
| Conchology | The branch of zoology dealing with the shells of mollusks |
| Cosmogony | The science of the nature of heavenly bodies |
| Cosmography | The science that describes and maps the main features of the |
| | universe |
| Cryobiology | The science that deals with the study of organisms, especially |
| | warmblooded animals, at low temperature. The principal effect of |
| | cold on living tissues is destruction of life or preservation of it at a |
| | reduced level of activity |
| Crystallography | The study of the structure, forms and properties of crystals |
| Cryogenics | The science dealing with the production, control and application of |
| | very low temperatures |
| Cryotherapy | Use of cold, but not freezing cold, as a form of treatment. |
| | Hypothermia may be deliberately induced during surgery, for |
| | instance, to decrease a patient's oxygen requirement |
| Cytochemistry | The branch of cytology dealing with the chemistry of cells |
| Cytogenetics | The branch of biology dealing with the study of heredity from the |
| | point of view of cytology and genetics |
| Cytology | The study of cells, especially their formation, structure and |
| | functions |
| Dactylography | The study of fingerprints for the purpose of identification |
| Dermatology | The study of skin and skin diseases |
| Ecology | The study of the relation of animals and plants to their |
| | surroundings, animate and inanimate |
| Econometrics | The application of mathematics in testing economic theories |
| Economics | The science dealing with the production, distribution and |
| | consumption of goods and services |
| Electronics | Studies the development, behaviour and applications of electronic |
| | devices and circuits |
| Electrostatics | It is a study of static electricity |
| Embryology | The study of development of embryos |
| Entomology | The study of insects |
| Epidemiology | The branch of medicine dealing with epidemic diseases |
| Epigraphy | The study of inscriptions |
| Ethnography | A branch of anthropology dealing with the scientific description of |
| | individual cultures |
| Ethnology | A branch of anthropology that deals with the origin, distribution |
| | and distinguishing characteristics of the races of mankind |
| Ethology | The study of animal behaviour |
| Eugenics | The study of the production of better offspring by the careful |
| | selection of parents |
| Fractography | A study of fractures in metal surfaces |
| Genealogy | The study of family origins and history. It includes the |
| | compilation of lists of ancestors and arranging them in pedigree |
| | charts |
| Genecology | The study of genetical composition of plant population in relation |
| | to their habitats |
| Genesiology | The science of generation |
| Genetics | The branch of biology dealing with the phenomena of heredity and |
| | the laws governing it |
| Geobiology | The biology of terrestrial life |
| Geobotany | The branch of botany dealing with all aspects of relations between |
| | plants and the earth's surface |
| Geochemistry | The study of the chemical composition of the earth's crust and the |
| | changes which take place within it |
| Geodesy | Methods of surveying the earth for making maps and correlating |
| | geological, gravitational and magnetic measurements. It is a |
| | branch of geo-physics |
| Geography | The development of science of the earth's surface, physical |
| | features, climate, population, etc |
| Geology | The science that deals with the physical history of the earth |
| Geomedicine | The branch of medicine dealing with the influence of climate and |
| | environmental conditions on health |
| Geomorphology | The study of the characteristics, origin and development of land |
| | forms |
| Geophysics | The physics of the earth |
| Gerontology | The study of old age, its phenomena, diseases, etc |
| Glaciology | The study of ice and the action of ice in all its forms, and therefore |
| | including now |
| Gynaecology | A study of diseases of women's reproductive organs |
| Histology | The study of tissues |
| Horticulture | The cultivation of flowers, fruits, vegetables and ornamental plants |
| Hydrodynamics | The mathematical study of the forces, energy and pressure of |
| | liquid in motion |
| Hydrography | The science of water measurements of the earth with special |
| | reference to their use for navigation |
| Hydrology | The study of water with reference to its occurrence and properties |
| | in the hydrosphere and atmosphere |
| Hydrometallurgy | The process of extracting metals at ordinary temperature by |
| | bleaching ore with liquids |
| Hydrometeorology | The study of the occurrence, movement and changes in the state of |
| | water in the atmosphere |
| Hydropathy | The treatment of disease by the internal and external use of water |
| Hydroponics | The cultivation of plants by placing the roots in liquid nutrient |
| | solutions rather than in soil |
| Hydrostatics | The mathematical study of forces and pressures in liquids |
| Hygiene | The science of health and its preservation |
| Limnology | The study of lakes |
| Lithology | It deals with systematic description of rocks |
| Mammography | Radiography of the mammary glands |
| Metallography | The study of the crystalline structures of metals and alloys |
| Metallurgy | The process of extracting metals from their ores |
| Meteorology | The science of the atmosphere and its phenomena |
| Metrology | The scientific study of weights and measures |
| Microbiology | The study of minute living organisms, including bacteria, molds |
| | and pathogenic protozoa |
| Molecular biology | The study of the structure of the molecules which are of |
| | importance in biology |
| Morphology | The science of organic forms and structures |
| Mycology | The study of fungi and fungus diseases |
| Neurology | The study of the nervous system, its functions and its disorders |
| Neuropathology | The study of diseases of the nervous system |
| Nosology | The classification of diseases |
| Numerology | The study of numbers. The study of the date and year of one's birth |
| | and to determine the influence on one's future life |
| Odontology study | The scientific |
| of the teeth | |
| Optics | The study of nature and properties of light |
| Ornithology | The study of birds |
| Orthopedics | The science of prevention, diagnosis and treatment of diseases and |
| | abnormalities of musculoskeletal system |
| Osteology | The study of the bones |
| Osteopathy | A therapeutic system based upon detecting and correcting faulty |
| | structure |
| Otology | The study of the ear and its diseases |
| Otorhinolaryngology | Study of diseases of ear, nose and throat |
| Paleobotany | The study of fossil plants |
| Paleontology | The study of fossils |
| Pathology | The study of diseases |
| Pharyngology | The science of the pharynx and its diseases |
| Phenology | The study of periodicity phenomena of plants |
| Philology | The study of written records, their authenticity, etc |
| Phonetics | The study of speech sounds and the production, transmission, |
| | reception, etc |
| Photobiology | The branch of biology dealing with the effect of light on |
| | organisms |
| Phrenology | The study of the faculties and qualities of minds from the shape of |
| | the skull |
| Phthisiology | The scientific study of tuberculosis |
| Phycology | The study of algae |
| Physical Science | The study of natural laws and processes other than those peculiar |
| | to living matters, as in physics, chemistry and astronomy |
| Physics | The study of the properties of matter |
| Physiography | The science of physical geography |
| Physiology | The study of the functioning of the various organs of living beings |
| Phytogeny | The science dealing with origin and growth of plants |
| Planetology | A study of the planets of the Solar System |
| Pomology | The science that deals with fruits and fruit growing |
| Psychology | The study of human and animal behaviour |
| Radio Astronomy | The study of heavenly bodies by the reception and analysis of the |
| | radio frequency electro-magnetic radiations which they emit or |
| | reflect |
| Radiobiology | The branch of biology which deals with the effects of radiations |
| | on living organisms |
| Radiology | The study of X-rays and radioactivity |
| Rheology | The study of the deformation and flow of matter |
| Seismology | The study of earthquakes and the phenomena associated with it |
| Selenology | The scientific study of moon, its nature, origin, movement, etc |
| Sericulture | The raising of silkworms for the production of raw silk |
| Sociology | The study of human society |
| Spectroscopy | The study of matter and energy by the use of spectroscope |
| Statistics | The collection and analysis of numerical data |
| Tectonics | Study of structural features of earth's crust |
| Teleology | The study of the evidences of design or purpose in nature |
| Telepathy | Communication between minds by some means other than sensory |
| | perception |
| Therapeutics | The science and art of healing |
| Topography | A special description of a part or region |
| Toxicology | The study of poisons |
| Virology | The study of viruses |
| Zoogeography | The study of the geological distributions of animals |
| Zoology | The study of animal life |

All these identifies can be repeatedly done to confirm the last identification is correct, there is not contingency.

The hardware configuration of automatic identification machine can refer FIG. 1A, FIG. 1B, FIG. 1C, FIG. 1D, FIG. 1E, FIG. 1F, FIG. 1G, FIG. 1H, FIG. 1i, FIG. 1J, FIG. 1K.

The operating method of the present invention can refer to FIG. 2A to FIG. 2F, FIG. 10, FIG. 11A, FIG. 11B, FIG. 12A, FIG. 12B, FIG. 13, FIG. 14, and FIG. 3A to FIG. 3E of the drawings.

The special PLC algorithms refers to FIG. 15 A, FIG. 15 B, FIG. 15 C, FIG. 15 D, FIG. 15E.

FIG. 4 can explain why the reason the method of identification of the present invention can get accurate result.

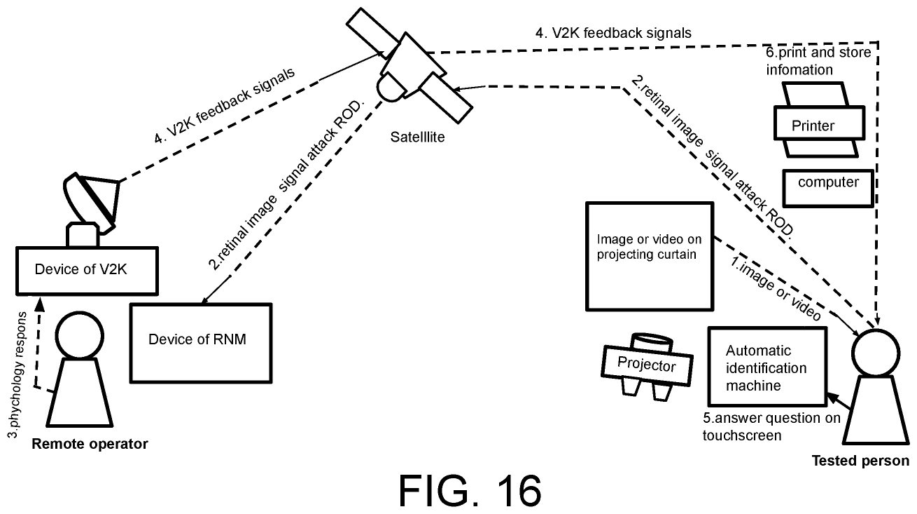

Automatic Identification Machine including tested person VS Device of V2K and Device of Remote Neural Monitor Technology refer FIG. 16.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What claims is:

1. An automatic machine to identify victim of abuse and remote operator using voice to skull and remote neural monitoring, comprising:
a projecting system for providing visual display, which comprises a picture and video database;
an operating electric box which comprises a box body, a power input arranged for connecting to a power source, a processor connected to said power input, and a touchscreen communicating with said processor; and
a polygraph meter,
wherein said automatic machine comprises a method to identify victim of abuse and remote operator using voice to skull and remote neural monitoring, executed through said processor of said operating electric box in connection to said projecting system and said polygraph meter, said method comprises the steps of:
visually displaying pictures and videos of a database (a) of said picture and video database such that a test person can select one disgusting item stored in said database (a), wherein said disgusting item is classified and determined by a PLC algorithm, only causes a remote operator to have disgusting psychological response but does not cause the tested person to have disgusting psychological response,
launching a retinal image signal psychological attacking ROD by instructing the tested person to watch the picture or video;
assigning test questions based on the retinal image signal psychological attack and obtaining answers from the tested person in response to the test questions through said touchscreen of said automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (a).

2. The automatic machine to identify victim of abuse and remote operator using voice to skull and remote neural monitoring according to claim 1, wherein said method to identify victim of abuse and remote operator using voice to skull and remote neural monitoring comprises the steps of:
visually displaying pictures and videos of a database (b) in said projecting system, wherein said database (b) consists of funny items,
launching a second retinal image signal psychological attacking ROD by instructing the tested person to select and watch one image or one video of funny item which only causes the remote operator to have funny psychological response but does not cause the tested person to have funny psychological response,
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (b).

3. The automatic machine to identify victim of abuse and remote operator using voice to skull and remote neural monitoring according to claim 2, wherein said method to identify victim of abuse and remote operator using voice to skull and remote neural monitoring comprises the steps of:
visually displaying pictures and videos of a database (e) in said projecting system, wherein said database (e) consists of foreign terrorist organizations items, wherein the foreign terrorist organizations items comprise wordings or images of accusing remote operator belonging to the foreign terrorist organization items which only causes the remote operator to have foreign terrorist organizations psychological response but does not cause the tested person to have foreign terrorist organizations psychological response;
launching a third retinal image signal psychological attacking ROD by instructing the tested person to select and watch one image or video of foreign terrorist organizations,
assigning test questions based on the psychological attack and obtaining answers from the tested person in response to the test questions through said touchscreen of the automatic machine, wherein the psychological response is angry psychological response and strong deny,
then using said polygraph meter to confirm that the test person is honest to finish the identification steps in said database (e).

4. The automatic machine to identify victim of abuse and remote operator using voice to skull and remote neural monitoring according to claim 3, wherein said method to identify victim of abuse and remote operator using voice to skull and remote neural monitoring comprises the steps of:
processing results obtained from the identification process by using the database (a), the database (b), and the database (e) and determining if the tested person is a victim of abuse;
if the test person is a victim of abuse, displaying the result on said touchscreen and printing a certificate of victim to the tested person through a printing system;
storing the results obtained from the identification process with a standardized form in a database in a computer; and
providing remote access to a user over a network so the user can update the information.

5. The automatic machine to identify victim of abuse and remote operator using voice to skull and remote neural monitoring according to claim 4, wherein said method to identify victim of abuse and remote operator using voice to skull and remote neural monitoring comprises the steps of:
visually displaying pictures and videos of a database (c) in said projecting system, wherein said database (c) consists of anti-political trend and anti-religious belief items, wherein the anti-political trend and anti-religious belief items only cause the remote operator to have anti-political trend and anti-religious belief psychological response and do not cause the tested person to have anti-political trend and anti-religious belief psychological response;
launching a fourth retinal image signal psychological attacking remote operator by instructing the tested person to select and watch one image or video of anti-political trend and anti-religious belief item;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (c),
automatically processing the results and printing and storing the political ideology and religious belief of the remote operator with a standardized form in a database in the computer for providing remote access to the user over the network so that the user can update the information.

6. The automatic machine to identify victim of abuse and remote operator using voice to skull and remote neural monitoring according to claim 4, wherein said method to identify victim of abuse and remote operator using voice to skull and remote neural monitoring comprises the steps of:
visually displaying pictures and videos of a database (d) in said projecting system, wherein said database (d) consists of countries and races items, wherein the countries and races items only cause the remote operator to have countries and races psychological response and do not cause the tested person to have countries and races psychological response;
launching a fifth retinal image signal psychological attacking remote operator by instructing the tested person to select and watch one image or video of countries and races items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (d),
automatically processing the results and printing and storing the countries and races of the remote operator with a standardized form in a database in the computer for providing remote access to the user over the network so that the user can update the information.

7. The automatic machine to identify victim of abuse and remote operator using voice to skull and remote neural monitoring according to claim 5, wherein said method to identify victim of abuse and remote operator using voice to skull and remote neural monitoring comprises the steps of:
visually displaying pictures and videos of a database (d) in said projecting system, wherein said database (d) consists of countries and races items, wherein the countries and races items only cause the remote operator to have countries and races psychological response and do not cause the tested person to have countries and races psychological response;
launching a fifth retinal image signal psychological attacking remote operator by instructing the tested person to select and watch one image or video of countries and races items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (d),
automatically processing the results and printing and storing the countries and races of the remote operator with a standardized form in a database in the computer for providing remote access to the user over the network so that the user can update the information.

8. The automatic machine to identify victim of abuse and remote operator using voice to skull and remote neural monitoring according to claim 4, wherein said method to identify victim of abuse and remote operator using voice to skull and remote neural monitoring comprises the steps of:
visually displaying pictures and videos of a database (f) in said projecting system, wherein said database (f) consists of languages and dialects items, wherein the languages and dialects items only cause the remote operator to have languages and dialects psychological response and do not cause the tested person to have languages and dialects psychological response;
launching a sixth retinal image signal psychological attacking remote operator by instructing the tested person to select and watch one image or video of languages and dialects items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (f).

9. The automatic machine to identify victim of abuse and remote operator using voice to skull and remote neural monitoring according to claim 5, wherein said method to identify victim of abuse and remote operator using voice to skull and remote neural monitoring comprises the steps of:
visually displaying pictures and videos of a database (f) in said projecting system, wherein said database (f) consists of languages and dialects items, wherein the languages and dialects items only cause the remote operator to have languages and dialects psychological response and do not cause the tested person to have languages and dialects psychological response;
launching a sixth retinal image signal psychological attacking remote operator by instructing the tested person to select and watch one image or video of languages and dialects items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (f).

10. The automatic machine to identify victim of abuse and remote operator using voice to skull and remote neural monitoring according to claim 6, wherein said method to identify victim of abuse and remote operator using voice to skull and remote neural monitoring comprises the steps of:
visually displaying pictures and videos of a database (f) in said projecting system, wherein said database (f) consists of languages and dialects items, wherein the languages and dialects items only cause the remote operator to have languages and dialects psychological response and do not cause the tested person to have languages and dialects psychological response;
launching a sixth retinal image signal psychological attacking remote operator by instructing the tested person to select and watch one image or video of languages and dialects items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (f).

11. The automatic machine to identify victim of abuse and remote operator using voice to skull and remote neural monitoring according to claim 7, wherein said method to identify victim of abuse and remote operator using voice to skull and remote neural monitoring comprises the steps of:
visually displaying pictures and videos of a database (f) in said projecting system, wherein said database (f) consists of languages and dialects items, wherein the languages and dialects items only cause the remote operator to have languages and dialects psychological response and do not cause the tested person to have languages and dialects psychological response;
launching a sixth retinal image signal psychological attacking remote operator by instructing the tested person to select and watch one image or video of languages and dialects items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (f).

12. The automatic machine to identify victim of abuse and remote operator using voice to skull and remote neural monitoring according to claim 4, wherein said method to identify victim of abuse and remote operator using voice to skull and remote neural monitoring comprises the steps of:
visually displaying pictures and videos of a database (g) in said projecting system, wherein said database (g) consists of scientific knowledge items, wherein the scientific knowledge items only cause the remote operator to have scientific knowledge psychological response and do not cause the tested person to have scientific knowledge psychological response;
launching a seventh retinal image signal psychological attacking remote operator by instructing the tested person to select and watch one image or video of scientific knowledge items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (g),
automatically processing the results and printing and storing the scientific knowledge results of the remote operator with a standardized form in a database in the computer for providing remote access to the user over the network so that the user can update the information.

13. The automatic machine to identify victim of abuse and remote operator using voice to skull and remote neural monitoring according to claim 7, wherein said method to identify victim of abuse and remote operator using voice to skull and remote neural monitoring comprises the steps of:
visually displaying pictures and videos of a database (g) in said projecting system, wherein said database (g) consists of scientific knowledge items, wherein the scientific knowledge items only cause the remote operator to have scientific knowledge psychological response and do not cause the tested person to have scientific knowledge psychological response;
launching a seventh retinal image signal psychological attacking remote operator by instructing the tested person to select and watch one image or video of scientific knowledge items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (g).

14. The automatic machine to identify victim of abuse and remote operator using voice to skull and remote neural monitoring according to claim 10, wherein said method to identify victim of abuse and remote operator using voice to skull and remote neural monitoring comprises the steps of:
visually displaying pictures and videos of a database (g) in said projecting system, wherein said database (g) consists of scientific knowledge items, wherein the scientific knowledge items only cause the remote operator to have scientific knowledge psychological response and do not cause the tested person to have scientific knowledge psychological response;
launching a seventh retinal image signal psychological attacking remote operator by instructing the tested person to select and watch one image or video of scientific knowledge items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (g),
automatically processing the results and printing and storing the scientific knowledge results of the remote operator with a standardized form in a database in the computer for providing remote access to the user over the network so that the user can update the information.

15. The automatic machine to identify victim of abuse and remote operator using voice to skull and remote neural monitoring according to claim 11, wherein said method to identify victim of abuse and remote operator using voice to skull and remote neural monitoring comprises the steps of:
visually displaying pictures and videos of a database (g) in said projecting system, wherein said database (g) consists of scientific knowledge items, wherein the scientific knowledge items only cause the remote operator to have scientific knowledge psychological response and do not cause the tested person to have scientific knowledge psychological response;
launching a seventh retinal image signal psychological attacking remote operator by instructing the tested person to select and watch one image or video of scientific knowledge items;
assigning test questions based on the retina image signal psychological attack and obtaining answers from the tested person in response to the test questions through said touchscreen of the automatic machine with an advanced automatic identifying system, wherein said touchscreen shows a small green light “CORRECT” or a small red light “WRONG” to the tested person in response to one “YES” or “NO” response from the test person and guides the tested person gradually to a final accurate identification,
then using said polygraph meter to confirm that the test person is honest to finish the identification steps in said database (g).

16. The automatic machine to identify victim of abuse and remote operator using voice to skull and remote neural monitoring according to claim 1, wherein the polygraph meter is connected to a tested person and the programmable logical controller is communicating with a touch screen.

17. The automatic machine to identify victim of abuse and remote operator using voice to skull and remote neural monitoring according to claim 4, wherein the polygraph meter is connected to a tested person and the programmable logical controller is communicating with a touchscreen.

18. The automatic machine to identify victim of abuse and remote operator using voice to skull and remote neural monitoring according to claim 15, wherein the polygraph meter is connected to a tested person and the programmable logical controller is communicating with a touchscreen.
